(19) United States
(12) Patent Application Publication
(10) Pub. No.: US 2016/0039796 A1
(43) Pub. Date: Feb. 11, 2016

(54) BARETTIN AND DERIVATIVES THEREOF FOR MEDICAL USE, IN PARTICULAR FOR THE TREATMENT OF DISEASES RELATED TO OXIDATIVE STRESS OR INFLAMMATION, AND FOR PRESERVING OR WASHING ORGANS

(71) Applicant: ABC Bioscience AS, Tromsø (NO)

(72) Inventors: Jeanette Hammer Andersen, Tromsø (NO); Espen Hansen, Tromsø (NO); Karl- Erik Eilertsen, Kvaløya (NO); Karianne Fredenfeldt Lind, Tromsø (NO); Bjarne Østerud, Tromsø (NO); Tim Hofer, Oslo (NO)

(21) Appl. No.: 14/778,640

(22) PCT Filed: Apr. 5, 2014
(86) PCT No.: PCT/EP2014/056885

§ 371 Date: Sep. 21, 2015

(30) Foreign Application Priority Data
Apr. 5, 2013 (EP) 13162540.2

(51) Int. Cl. C07D 403/06

(52) U.S. Cl. CPC C07D 403/06

(57) ABSTRACT

The compounds of formula (I) herein include barettin and derivatives thereof, or any pharmaceutically acceptable salt thereof for use as a medicament. Further the compounds may be used for the treatment of diseases related to oxidative stress and the treatment of inflammatory diseases; for the cosmetic treatment of skin aging; in a solution for the preservation and/or washing of organs; and as a food and/or feed additive.

### TECHNICAL FIELD OF THE INVENTION

The present invention relates to the chemical compound barettin and derivatives thereof for use as a medicament. In particular the present invention relates to barettin and derivatives thereof for use in the treatment of diseases related to oxidative stress, such as cardiovascular disease, and barettin and derivatives thereof for the use in the treatment of inflammatory diseases. Another aspect is the use of barettin and derivatives thereof as a cosmetic agent for the treatment of skin aging and the use of barettin and derivatives as a food or feed additive.

### BACKGROUND OF THE INVENTION

Marine sponges are a rich source of bioactive compounds. The present inventors have isolated barettin (cyclo-[(6-bromo-8-en-tryptophan)-arginine] or alternatively N-{3[(6-Brom-1H-indol-3-ylmethylen)-3,6-dioxo-piperazin-2-yl]-propyl}-guanidin) and derivatives thereof from the marine sponge Geodia baretti collected of the coast of northern Norway. In 2002, data was published suggesting the structure of Barettin (Softer S. et al., tetrahedron letters, 2002, 43, 3385-3386). This was later confirmed with the successful synthesis of barettin (Johnson, A.-L. et al., tetrahedron 2004, 60, 961-965). Since then, the anti-fouling properties of barettin and derivatives have been disclosed in WO 03/081199, and it has been shown that barettin is a serotonin receptor ligand and that the serotonin-like structure of barettin is probably giving the molecule its anti-fouling properties (Hedner, E. et al., J. Nat. Prod. 2006, 69, 1421-1424).

The therapeutic potential of barettin and derivatives thereof has so far not been investigated. Hence, improved or alternative treatment of medical conditions using bioactive chemical compounds such as barettin or derivatives thereof would be advantageous, and in particular improved or alternative treatments of diseases related to oxidative stress, such as cardiovascular diseases, and inflammatory conditions would be advantageous. The present inventors have surprisingly found that the compounds of the present invention, i.e. barettin and derivatives thereof, have therapeutic potential and represent alternative and improved agents for medical use. Particularly, the compounds of the present invention have surprising anti-oxidative and anti-inflammatory properties.

### SUMMARY OF THE INVENTION

Thus, an object of the present invention relates to the provision of alternative or improved chemical compounds for use as medicaments.

In particular, it is an object of the present invention to provide chemical compounds such as barettin and derivatives thereofthat solves the above mentioned problems of the prior art with providing new compounds for medical use, particularly for use in the treatment of diseases related to oxidative stress such as cardiovascular disease and also inflammatory conditions.

Thus, one aspect of the invention relates to a compound of formula (I)

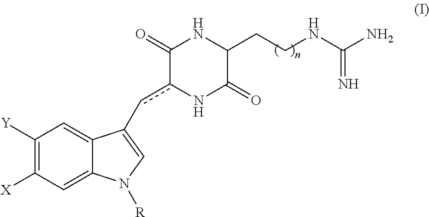

wherein

X and Y are independently selected from the group consisting of hydrogen and a halogen,

[symbol] denotes a single bond or a double bond,

R is selected from the group consisting of hydrogen and C₁-C₆ alkyl, and

n is an integer selected from 1, 2, 3 and 4;

or any pharmaceutically acceptable salt thereof for use as a medicament.

Another aspect of the present invention relates to a compound of formula (I)

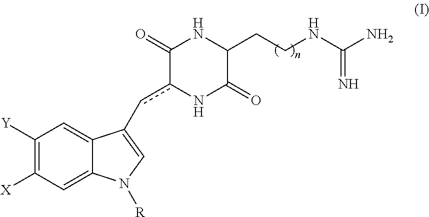

wherein

X and Y are independently selected from the group consisting of hydrogen and a halogen,

[symbol] denotes a single bond or a double bond,

R is selected from the group consisting of hydrogen and C₁-C₆ alkyl, and

n is an integer selected from 1, 2, 3 and 4;

or any pharmaceutically acceptable salt thereof for use in treatment of diseases related to oxidative stress.

Yet another aspect of the present invention is to provide a compound of formula (I)

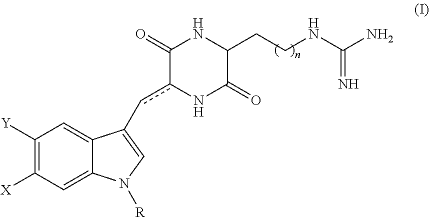

wherein

X and Y are independently selected from the group consisting of hydrogen and a halogen,

[symbol] denotes a single bond or a double bond,

R is selected from the group consisting of hydrogen and C₁-C₆ alkyl, and n is an integer selected from 1, 2, 3 and 4;

or any pharmaceutically acceptable salt thereof for use in treatment of inflammatory diseases.

Yet another aspect is the use of the compound of formula (I) or any pharmaceutically acceptable salt thereof in a solution for the preservation and/or washing of organs.

Still another aspect of the present invention is the use of a compound of formula (I) for the cosmetic treatment of skin aging.

A final aspect of the present invention is the use of a compound of formula (I) as a food and/or feed additive.

### BRIEF DESCRIPTION OF THE FIGURES

FIG. 1 shows the tested compounds barettin (MBC-005/MBC-011 isolated and synthesized respectively) and de-bromo barettin (MBC-007). Alkyl and guanidine protons are not shown, but are implicit,

FIG. 2 shows the antioxidant effect of MBC-005 and MBC-007 in the ferric reducing antioxidant power (FRAP) assay. Both molecules reduces iron in a dose-dependent manner. Data is from 1 representative experiment, n=2,

FIG. 3 shows how MBC-005 and MBC-007 act in a dose-dependent manner in the oxygen radical absorbance capacity (ORAC) assay to protect fluorescein from degradation. Results shown are from one representative experiment, n=2,

FIG. 4 shows cellular lipid peroxidation antioxidant activity (CLPAA) results for MBC-005 and MBC-007. MBC-005 acts in a dose-dependent manner to reduce lipid peroxidation in HepG2 cells. This effect is not seen with MBC-007 for this particular assay. BHT (10 μM) was used as a comparative control. Results are normalized to positive control CumOOH, n=3,

FIG. 5 shows the anti-inflammatory effect of synthesized barettin (here designated MBC-011). The graph shows the percent inhibition of tumor necrosis factor-alpha (TNF-α) and Interleukin-1 beta (IL-1β) in LPS induced monocytes (THP-1 cells),

FIG. 6 shows results from cytotoxicity testing of the MBC-005 and MBC-007 compounds using HepG2 cells. Results are expressed as percentage survival after 24 h exposure. Presented results are from one representative experiment, n=3,

FIG. 7 shows results from cytotoxicity testing of the MBC-005 and MBC-007 compounds using MRC5 cells. Results are expressed as percentage survival after 24 h exposure. Presented results are from one representative experiment, n=3,

FIG. 8A shows total cholesterol (mmol/L) and LDL cholesterol (mmol/L) from different groups of the feeding experiment after 7 weeks,

FIG. 8B shows Triacylglycerol (mmol/L) and OxLDL (ng/mL) from different groups of the feeding experiment after 7 weeks,

FIG. 9 a) shows the lesion area in the aortic arch (relevant aorta area as depicted in b) in the upper part of the picture),

FIG. 10 a) shows the lesion area in the descending aorta (relevant aorta area as depicted in b) in the middle part of the picture),

FIG. 11 a) shows the lesion area in the infrarenal part of aorta (relevant area as depicted in b) in the lower part of the picture),

FIG. 12 a) shows the lesion area in the total aorta (total aorta depicted in b)),

FIG. 13 shows the effect of barettin on LPS-induced release of IL-6 from THP-1 cells (n=6). ** P<0.01; *** P<0.001 compared to LPS treated control (without barettin or dexamethasone; one-way ANOVA with Dunnett's post-test).

The present invention will now be described in more detail in the following.

### DETAILED DESCRIPTION OF THE INVENTION

### Definitions

Prior to discussing the present invention in further details, the following terms and conventions will first be defined:

Herein “C₁-C₆ alkyl” is defined as an alkyl moiety having 1 to 6 carbon atoms. The alkyl moiety may be linear or branched. Similarly “C₁-C₃ alkyl” is defined as an alkyl moiety having 1 to 3 carbon atoms. The alkyl moiety may be linear or branched. Linear alkyls include —CH₃, —CH₂CH₃, —CH₂CH₂CH₃, —CH₂CH₂CH₂CH₃, —CH₂CH₂CH₂CH₂CH₃, and —CH₂CH₂CH₂CH₂CH₂CH₃. Branched alkyls may include but are not limited to —CH(CH₃)₂, —C(CH₃)₃, —CH(CH₂CH₃)₂, —CH(CH₃)CH₂CH₃.

Herein “hydrogen” is defined as the moiety —H, also sometimes referred to as a proton.

Herein “halogen” is defined as an element stemming from group 17 in the periodic table consisting of five chemically related elements, fluorine (F), chlorine (Cl), bromine (Br), iodine (I), and astatine (At). Astatine is however rarely used in organic chemistry, and thus in the present context halogen may be said to refer to any one of a fluorine, chlorine, bromine and iodine.

Herein “pharmaceutically acceptable salt” is defined as a ionic species, wherein for example one or more protons has been removed from a compound to form a negatively charged species and another positively charged ionic species is bound to the negatively charged species to form a neutral salt. Alternatively one or more protons are added to a compound to form a positively charged species and another negatively charged ionic species is bound to the positively charged species to form a neutral salt. By neutral salt is meant a salt with a zero net charge. The formed salt fulfills the pharmacological requirements for a medicament, for example it does not have intolerable toxicity or dissolution characteristics, and it is therefore characterized as pharmaceutically acceptable. Such salts may be selected from the group consisting of but not limited to: alkali metal salts including lithium, sodium, and potassium salts, alkaline earth metal salts including magnesium, calcium, and strontium salts, metal salts including aluminium and zinc salts, acid salts including e.g. hydrochloride salts or hydrobromide salts, and more complex salts such as for example nitrate, sulfate, bisulfate, phosphate, acid phosphate, isonicotinate, acetate, lactate, salicylate, citrate, tartrate, pantothenate, bitartrate, ascorbate, succinate, maleate, gentisinate, fumarate, gluconate, glucaronate, saccharate, formate, benzoate, glutamate, methanesulfonate, ethanesulfonate, benzensulfonate, p-toluenesulfonate, pamoate salts, and any combination of said salts.

Herein “diseases related to oxidative stress” is defined as a disease or medical condition wherein the onset or progression of the disease is promoted by oxidative stress. Oxidative stress is caused by an inbalance in the body where more oxidative species are present that anti-oxidative species. Particularly, “reactive oxidative species” (ROS) are considered harmful in this regard. Since oxidative stress is believed to be responsible for the pathogenesis of many neurological, cardiovascular, malignant and age-associated diseases, the present invention contemplates all such diseases. “Diseases related to oxidative stress” may alternatively be designated “oxidative stress”.

Herein “inflammatory diseases” is defined as any diseases caused by an inflammation. Inflammation is part of the biological response of vascular tissues to harmful stimuli, including pathogens, damaged cells, or irritants. Inflammatory diseases may alternatively be designated as “inflammation” or “inflammatory conditions”. There are a large number of types of inflammatory diseases, depending on which part of the body is affected by the condition. A few examples of inflammatory diseases include, but are not limited to appendicitis, Bursitis, Colitis, Cystitis, Dermatitis, Meningitis, Phlebitis, Rhinitis, Tendonitis, Tonsillitis, Vasculitis, and Arthritis, and inflammatory cardiovascular diseases.

The present inventors have surprisingly identified a therapeutic potential of the compound Barettin and derivatives thereof. A number of in vitro and in vivo experiments performed by the inventors have demonstrated a potential for barettin in the treatment of diseases related to oxidative stress and inflammatory conditions, while at the same time showing that the cytotoxicity of these compounds is not present or negligible within the dose ranges used in the experiments showing anti-oxidative and anti-inflammatory effects in vitro. Thus, a first aspect of the present invention is a compound of formula (I)

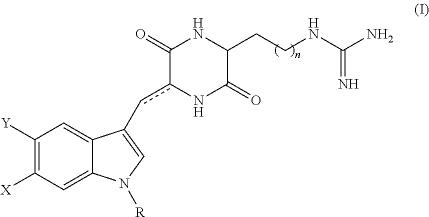

wherein

X and Y are independently selected from the group consisting of hydrogen and a halogen,

[symbol] denotes a single bond or a double bond,

R is selected from the group consisting of hydrogen and C₁-C₆ alkyl, and

n is an integer selected from 1, 2, 3 and 4;

or any pharmaceutically acceptable salt thereof for use as a medicament.

In one preferred embodiment X is halogen and Y is independently selected from the group consisting of hydrogen and a halogen. In another preferred embodiment X is a halogen and Y is hydrogen. In another embodiment the halogen may be selected from the group consisting of fluorine, chlorine, bromine and iodine. Most preferably the halogen is bromine.

C₁-C₆ alkyl may include methyl, ethyl, propyl, n-butyl, n-pentyl, n-hexane, and any branched derivatives having up to 6 carbons including isopropyl, and tert-butyl. In yet another preferred embodiment R is selected from the group consisting of hydrogen and C₁-C₃ alkyl. C₁-C₃ alkyl may include methyl, ethyl, propyl, and isopropyl. Even more preferably R is hydrogen.

In another preferred embodiment n is 2. This corresponds to the length of the relevant carbon chain in barettin corresponding to the side chain of the amino acid arginine. Finally, in another preferred embodiment [symbol] denotes a double bond. Again this corresponds to the double bond as present in barettin.

Due to the double bond present in the compound of formula (I) wherein [symbol] denotes a double bond this compound may exist in a Z and a E isomeric form. In a preferred embodiment said compound is a mixture of the Z and E isomer. In another interesting embodiment said compound is the E isomer. In yet another preferred embodiment said compound is the Z isomer.

In a highly preferred embodiment X is bromine and Y is hydrogen, [symbol] denotes a double bond, R is hydrogen, and n is 2. In an even more preferred embodiment the compound of formula (I) is barettin, i.e. corresponding to the compound of formula (II):

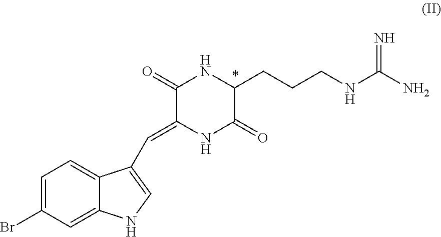 [SMILES: N=C(N)NCCCC1NC(=O)/C(=C/c2c[nH]c3cc(Br)ccc23)NC1=O]

When the compound of the invention includes a double bond and particularly when it is barettin as defined in formula (II) the compound is preferably the mixture of the Z and E isomer as obtained when isolating the compound from the marine sponge Geodia baretti. Even more preferably the compound corresponds to the major isomer of the compound as obtained when isolating the compound from the marine sponge Geodia baretti. In an alternative embodiment the compound corresponds to the minor isomer of the compound as obtained when isolating the compound from the marine sponge Geodia baretti. For barettin the Z and E isomers are separable by HPLC. Thus, the compound of the invention may preferably be selected from the group consisting of Z-barettin, E-barettin or any mixture thereof. Preferably the compound of the invention is Z-barettin. Generally, when referring to a given isomer or enantiomer of the compounds of the present invention, this means that the isomer or enantiomer is substantially free of the other isomer and/or enantiomer, or alternatively essentially free of the other isomer and/or enantiomer. Thus, the isomer or enantiomer may be 90% single isomer and/or enantiomer, such as 91%, 92%, 93%, 94%, 95%, 96%, 97%, 98%, 99%, 99.5%, 99.8%, preferably 99.9% single sourer and/or enantiomer.

Furthermore, in the compound of formula (I) wherein [symbol] denotes a double bond this compound comprises one enantiomeric centre where the arginine side chain is attached to the heterocyclic ring. This position is marked by a “*” in the compound of formula (II) to illustrate the general position of this centre in all the compounds herein, wherein [symbol] denotes a double bond.

Thus, the compound of formula (I) may preferably be a mixture of enantiomers. The compound may preferably be the racemate. In another embodiment the compound of formula (I) may be the (S)-enantiomer. Alternatively it may be the (R)-enantiomer. In a highly preferred embodiment the compound is the compound barettin of formula (II) and the enantiomer or mixture of enantiomers is that obtained when isolating barettin from the marine sponge Geodia baretti. Even more preferably it is the enantiomer or mixture of enantiomers obtained when isolating the major (Z/E) isomer of barettin from the marine sponge Geodia baretti. In one embodiment the compound may be selected from the group consisting of (Z, (S))-barettin, (Z, (R))-barettin, (E, (S))-barettin and (E, (R))-barettin. In a highly preferred embodiment the compound of the invention is (Z, (S))-barettin.

In another embodiment of the present invention the compound of formula (I) is a compound wherein [symbol] denotes a single bond. In this embodiment Z and E isomer do not exist, however the compounds now include two enantiomeric centres since the ring-carbon of the single bond now introduced is also an enantiomeric centre. Thus these compounds of formula (I) wherein [symbol] denotes a single bond may include four epimers, i.e. the (S,S), the (R,R), the (R,S) and the (S,R) form of the compounds. In a preferred embodiment the compound offormula (I) wherein [symbol] denotes a single bond is (8,9)-dihydrobarettin, including any epimers thereof.

The compounds of the present invention were investigated for anti-oxidant effects in several in vitro assays, including for example Ferric reducing antioxidant power (FRAP) assay, a cellular lipid peroxidation anti-oxidant activity (CLPAA) assay, and oxygen radical absorbance capacity (ORAC) assays. The compounds displayed significant potential in these assays.

Therefore, a second aspect of the present invention is a compound of formula (I)

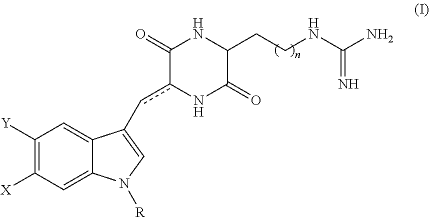

wherein

X and Y are independently selected from the group consisting of hydrogen and a halogen,

[symbol] denotes a single bond or a double bond,

R is selected from the group consisting of hydrogen and C₁-C₆ alkyl, and

n is an integer selected from 1, 2, 3 and 4;

or any pharmaceutically acceptable salt thereof for use in treatment of diseases related to oxidative stress.

Diseases related to oxidative stress may be selected from the group consisting of Cancer, Cardiovascular diseases, Motor neuron diseases, atherosclerosis, heart failure, myocardial infarction, Schizophrenia dementia, Bipolar disorder, fragile X syndrome, Sickle Cell Disease, lichen planus, vitiligo, autism, cataract, diabetes, diabetic vasculapathy, chronic fatigue syndrome, and neurodegenerative diseases.

In a particularly preferred embodiment the disease related to oxidative stress is cardiovascular disease. Cardiovascular disease may preferably be selected from the group consisting of Coronary heart disease, (also ischaemic heart disease or coronary artery disease), Cardiomyopathy, Hypertensive heart disease, Heart failure, Cor pulmonale, Cardiac dysrhythmias, Inflammatory heart disease, Endocarditis, Inflammatory cardiomegaly, Myocarditis, Valvular heart disease, Stroke, cerebrovascular disease, and Peripheral arterial disease.

Neurodegenerative diseases may be selected from the group consisting of Parkinsons disease, multiple sclerosis, ALS, multi-system atrophy, Alzheimer's disease, Huntington's disease, stroke, and Pick's disease.

The disease related to oxidative stress is preferably selected from the group consisting of Cancer, Cardiovascular disease, Parkinson's disease, Motor neuron disease, Huntington's disease, Atherosclerosis, Myocardial infarction, Bipolar disorder, Fragile X syndrome, Sickle Cell Disease, Lichen Planus, Vitiligo, Autism, and Chronic Fatigue Syndrome.

The cardiovascular disease is preferably selected from the group consisting of Coronary heart disease, Cardiomyopathy, Hypertensive heart disease, Cor pulmonale, Inflammatory heart disease, Endocarditis, Inflammatory cardiomegaly, Myocarditis, Valvular heart disease, Stroke, cerebrovascular disease, and Peripheral arterial disease.

In another embodiment said disease related to oxidative stress is preferably cancer. Cancers may include Carcinomas, Sarcomas, Lymphomas and leukemias, Germ cell tumors, and Blastomas. Specifically, cancers may be selected from the group consisting of Acute lymphoblastic leukemia, Acute myeloid leukemia, Adrenocortical carcinoma, AIDS-related cancers, AIDS-related lymphoma, Anal cancer, Appendix cancer, Astrocytoma, Basal cell carcinoma, Bile duct cancer, Bladder cancer, Bone cancer, Osteosarcoma/Malignant fibrous histiocytoma, Brainstem glioma, Brain cancer, Brain tumor—cerebellar astrocytoma, Brain tumor—cerebral astrocytoma/malignant glioma, Brain tumor—ependymoma, Brain tumor—medulloblastoma, Brain tumor—supratentorial primitive neuroectodermal tumors, Brain tumor—visual pathway and hypothalamic glioma, Breast cancer or Breast carcinoma, Bronchial adenomas/carcinoids, Burkitt lymphoma, Carcinoid tumor, Carcinoid tumor, gastrointestinal, Carcinoma of unknown primary, Central nervous system lymphoma, Cerebellar astrocytoma, Cerebral astrocytoma or Malignant glioma, Cervical cancer, Chronic lymphocytic leukemia, Chronic myelogenous leukemia, Chronic myeloproliferative disorders, Colon Cancer or Colon carcinoma, Cutaneous T-cell lymphoma, Desmoplastic small round cell tumor, Endometrial cancer, Ependymoma, Esophageal cancer, Ewing's sarcoma, extracranial germ cell tumor, Extragonadal Germ cell tumor, Extrahepatic bile duct cancer, Eye Cancer—Intraocular melanoma, Eye Cancer—Retinoblastoma, Gallbladder cancer, Gastric (Stomach) cancer, Gastrointestinal Carcinoid Tumor, Gastrointestinal stromal tumor (GIST), Germ cell tumor—extracranial, extragonadal, or ovarian, Gestational trophoblastic tumor, Glioma of the brain stem, Glioma—Childhood Cerebral Astrocytoma, Glioma—Childhood Visual Pathway and Hypothalamic, Gastric carcinoid, Head and neck cancer, Heart cancer, Hepatocellular (liver) cancer, Hodgkin lymphoma, Hypopharyngeal cancer, Islet Cell Carcinoma (Endocrine Pancreas), Kaposi sarcoma, Kidney cancer (renal cell cancer), Laryngeal Cancer, Leukemia—acute lymphoblastic (also called acute lymphocytic leukemia), Leukemia—acute myeloid (also called acute myelogenous leukemia), Leukemia—chronic lymphocytic (also called chronic lymphocytic leukemia), Leukemia—chronic myelogenous (also called chronic myeloid leukemia), Leukemia—hairy cell, Lip and Oral Cavity Cancer, Liposarcoma, Liver Cancer (Primary), Lung Cancer, Non-Small Cell Lung Cancer, Lymphoma, Burkitt, Lymphoma, cutaneous T-Cell, Lymphoma, Hodgkin, Lymphoma—Primary Central Nervous System, Macroglobulinemia, Waldenström, Malignant Fibrous Histiocytoma of Bone/Osteosarcoma, Medulloblastoma, Melanoma, Melanoma-Intraocular (Eye), Merkel Cell Carcinoma, Mesothelioma, Multiple Endocrine Neoplasia Syndrome, Multiple Myeloma/Plasma Cell Neoplasm, Mycosis Fungoides, Myelodysplastic Syndromes Myelodysplastic/Myeloproliferative Diseases, Nasal cavity and paranasal sinus cancer, Nasopharyngeal carcinoma, Neuroblastoma, Oropharyngeal cancer, Ovarian cancer, Ovarian epithelial cancer (Surface epithelial-stromal tumor), Ovarian germ cell tumor, Pancreatic cancer, Pancreatic cancer—islet cell, Parathyroid cancer, Penile cancer, Pharyngeal cancer, Pheochromocytoma, Pineal astrocytoma, Pineal germinoma, Pineoblastoma and supratentorial primitive neuroectodermal tumors, Pituitary adenoma, Plasma cell neoplasia/Multiple myeloma, Pleuropulmonary blastoma, Primary central nervous system lymphoma, Prostate cancer, Rectal cancer, Renal cell carcinoma (kidney cancer), Retinoblastoma, Rhabdomyosarcoma, Salivary gland cancer, Sarcoma—soft tissue, Sarcoma—uterine, Sézary syndrome, Skin cancer (nonmelanoma), Skin carcinoma—Merkel cell, Small cell lung cancer, Small intestine cancer, Soft tissue sarcoma, Supratentorial primitive neuroectodermal tumor, TT-Cell lymphoma, cutaneous, Testicular cancer, Throat cancer, Thymoma and Thymic carcinoma, Thyroid cancer, Trophoblastic tumor, gestational, Urethral cancer, Uterine cancer—endometrial, Uterine sarcoma, Vaginal cancer, Vulvar cancer, and Wilms tumor (kidney cancer, childhood).

In a preferred embodiment the treatment of diseases related to oxidative stress may be a prophylactic treatment. In the treatments of diseases related to oxidative stress the compounds of the present invention may preferably be co-administered with other Active Product Ingredients, in other words it may be used in combination therapy of diseases related to oxidative stress.

A preferred embodiment is the compound of formula (I) for use in treatment of oxidative stress in organs for organ transplantation, preferably during storage and/or transportation, preferably cold storage and/or transportation. Preferably, the compound of formula (I) is provided in a preservation and/or washing solution. Preferably the organ to be transplanted is immersed and/or washed in said solution. Preferably the preservation solution is for human organs. The organs may be any body part or internal organ but may preferably be selected from the group consisting of hearts, livers, kidneys, and pancreases.

The present inventors have also surprisingly found that the compounds of the invention display significant efficacy in in vitro anti-inflammatory assays. Thus, it has been demonstrated that barettin inhibits tumor necrosis factor-alpha (TNF-α) and Interleukin-1 beta (IL-1β) in LPS induced monocytes, such as THP-1 cells.

Therefore, a third aspect of the present invention is a compound of formula (I)

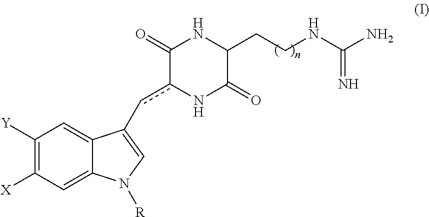

wherein

X and Y are independently selected from the group consisting of hydrogen and a halogen,

[symbol] denotes a single bond or a double bond,

R is selected from the group consisting of hydrogen and C₁-C₆ alkyl, and n is an integer selected from 1, 2, 3 and 4;

or any pharmaceutically acceptable salt thereof for use in treatment of inflammatory diseases.

A number of medical conditions are linked with chronic inflammation or have an inflammatory component, and thus said inflammatory diseases may preferably be selected from the group consisting of acid reflux/heartburn, acne, Acne vulgaris, allergies and hypersensitivities, Alzheimer's disease, ankylosing spondylitis, appendicitis, arthritis, asthma, atherosclerosis, autoimmune diseases, bronchitis, Bursitis, carditis, celiac disease, chronic pain, Chronic prostatitis, Colitis, Crohn's disease, cirrhosis, colitis, cystitis, dementia, dermatitis, diabetes, diverticulitis, dry eyes, edema, emphysema, eczema, fibromyalgia, gastroenteritis, gingivitis, Glomerulonephritis, heart disease, hepatitis (types A, B and C), high blood pressure, inflammatory cardiovascular diseases, Inflammatory bowel diseases, insulin resistance, interstitial cystitis, irritable bowel syndrome, joint pain, systemic lupus erythematous, meningitis, metabolic syndrome (syndrome X), myositis, nephritis, obesity, osteoarthritis, osteopenia, osteoporosis, Parkinson's disease, periodontal disease, Pelvic inflammatory disease, phlebitis, polyarteritis, polychondritis, psoriasis, psoriatic arthritis, Reperfusion injury, rheumatoid arthritis, Rhinitis, Sarcoidosis, scleroderma, sinusitis, Sjögren's syndrome, spastic colon, systemic candidiasis, tendonitis, tonsillitis, Transplant rejection, vaginitis, ulcerative colitis, and Vasculitis.

The inflammatory disease is preferably selected from the group consisting ofacid reflux/heartburn, acne, Acne vulgaris, allergies and hypersensitivities, ankylosing spondylitis, appendicitis, arthritis, asthma, atherosclerosis, autoimmune diseases, bronchitis, Bursitis, carditis, celiac disease, chronic pain, Chronic prostatitis, Colitis, Crohn's disease, cirrhosis, colitis, cystitis, dermatitis, diabetes, diverticulitis, dry eyes, edema, emphysema, eczema, fibromyalgia, gastroenteritis, gingivitis, Glomerulonephritis, heart disease, hepatitis, high blood pressure, inflammatory cardiovascular disease, Inflammatory bowel diseases, insulin resistance, interstitial cystitis, joint pain, systemic lupus erythematous, meningitis, metabolic syndrome (syndrome X), myositis, nephritis, obesity, osteoarthritis, osteopenia, osteoporosis, Parkinson's disease, periodontal disease, Pelvic inflammatory disease, phlebitis, polyarteritis, polychondritis, psoriasis, psoriatic arthritis, Reperfusion injury, rheumatoid arthritis, Rhinitis, Sarcoidosis, scleroderma, sinusitis, Sjögren's syndrome, spastic colon, systemic candidiasis, tendonitis, tonsillitis, Transplant rejection, vaginitis, ulcerative colitis, and Vasculitis.

The inflammatory disease is preferably atherosclerosis.

In a preferred embodiment the treatment of inflammatory diseases may be a prophylactic treatment. In the treatments of inflammatory diseases the compounds of the present invention may preferably be co-administered with other Active Product Ingredients, in other words it may be used in combination therapy of inflammatory diseases.

A preferred embodiment is the compound of formula (I) for use in treatment of inflammation in organs for organ transplantation, preferably during storage and/or transportation, preferably cold storage and/or transportation. Preferably, the compound of formula (I) is provided in a preservation and/or washing solution. Preferably, the organ to be transplanted is immersed and/or washed in said solution. Preferably the preservation solution is for human organs. The organs may be any body part or internal organ but may preferably be selected from the group consisting of hearts, livers, kidneys, and pancreases.

With respect to the second and third aspect of the present invention as described above the compound of formula (I) is subject to the same preferred embodiments as described for the first aspect of the present invention. Thus, in a preferred embodiment with respect to the compound of formula (I) or any pharmaceutically acceptable salt thereof for use in the treatment of diseases related to oxidative stress or inflammatory diseases, X is halogen and Y is independently selected from the group consisting of hydrogen and a halogen, in another embodiment X is halogen and Y is hydrogen. In another embodiment the halogen may be selected from the group consisting of fluorine, chlorine, bromine and iodine. Most preferably the halogen is bromine.

C₁-C₆ alkyl may include methyl, ethyl, propyl, n-butyl, n-pentyl, n-hexane, and any branched derivatives having up to 6 carbons including isopropyl, and tert butyl. In yet another preferred embodiment R is selected from the group consisting of hydrogen and C₁-C₃ alkyl. C₁-C₃ alkyl may include methyl, ethyl, propyl, and isopropyl. Even more preferably R is hydrogen.

I another preferred embodiment n is 2. This corresponds to the length of the carbon chain in barettin. Finally, in another preferred embodiment [symbol] denotes a double bond. Again this corresponds to the double bond as present in barettin.

Due to the double bond present in the compound of formula (I) wherein [symbol] denotes a double bond this compound may exist in a Z and a E isomeric form. In a preferred embodiment said compound is a mixture of the Z and E isomer. In another interesting embodiment said compound is the E isomer. In yet another embodiment said compound is the Z isomer.

In a highly preferred embodiment X is bromine and Y is hydrogen, [symbol] denotes a double bond, R is hydrogen, and n is 2. In an even more preferred embodiment the compound of formula (I) is barettin, i.e. corresponding to the compound of formula (II):

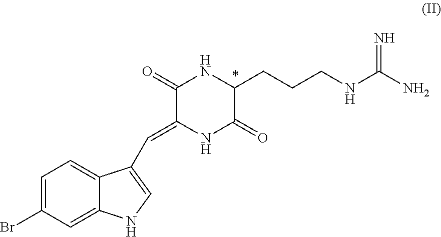 [SMILES: N=C(N)NCCCC1NC(=O)/C(=C/c2c[nH]c3cc(Br)ccc23)NC1=O]

When the compound of the invention includes a double bond and particularly when it is barettin as defined in formula (II) the compound is preferably the mixture of the Z and E isomer as obtained when isolating the compound from the marine sponge Geodia baretti. Even more preferably the compound corresponds to the major isomer of the compound as obtained when isolating the compound from the marine sponge Geodia baretti. In an alternative embodiment the compound corresponds to the minor isomer of the compound as obtained when isolating the compound from the marine sponge Geodia baretti. For barettin the Z and E isomers are separable by HPLC. Thus, the compound of the invention may preferably be selected from the group consisting ofZ-barettin, E-barettin or any mixture thereof. Preferably, the compound of the invention is Z-barettin. Generally, when referring to a given isomer or enantiomer of the compounds of the present invention, this means that the isomer or enantiomer is substantially free of the other isomer and/or enantiomer, or alternatively essentially free of the other isomer and/or enantiomer. Thus, the isomer or enantiomer may be 90% single isomer and/or enantiomer, such as 91%, 92%, 93%, 94%, 95%, 96%, 97%, 98%, 99%, 99.5%, 99.8%, preferably 99.9% single isomer and/or enantiomer.

Furthermore, in the compound of formula (I) wherein [symbol] denotes a double bond this compound comprises one enantiomeric centre where the arginine side chain is attached to the heterocyclic ring. This position is marked by a “*” in the compound of formula (II) to illustrate the general position of this centre in all the compounds herein, wherein [symbol] denotes a double bond.

Thus, the compound of formula (I) may preferably be a mixture of enantiomers. The compound may be the racemate. In another embodiment the compound of formula (I) may be the (S)-enantiomer. Alternatively it may be the (R)-enantiomer. In a highly preferred embodiment the compound is the compound barettin of formula (II) and the enantiomer or mixture of enantiomers is that obtained when isolating barettin from the marine sponge Geodia baretti. Even more preferably it is the enantiomer or mixture of enantiomers obtained when isolating the major (Z/E) isomer of barettin from the marine sponge Geodia baretti. In one embodiment the compound may be selected from the group consisting of (Z, (S))-barettin, (Z, (R))-barettin, (E, (S))-barettin and (E, (R))-barettin. In a highly preferred embodiment the compound of the invention is (Z, (S))-barettin.

In another embodiment of the present invention the compound of formula (I) is a compound wherein [symbol] denotes a single bond. In this embodiment Z and E isomer do not exist, however the compounds now include two enantiomeric centres since the ring-carbon of the single bond now introduced is also an enantiomeric centre. Thus these compounds of formula (I) wherein [symbol] denotes a single bond may include four epimers, i.e. the (S,S), the (R,R), the (R,S) and the (S,R) form of the compounds. In a preferred embodiment the compound of formula (I) wherein [symbol] denotes a single bond is (8,9)-dihydrobarettin, including any epimers thereof.

Another aspect of the present invention is a method of treating oxidative stress or inflammation in a mammal comprising administering a compound of formula (I) or any pharmaceutically acceptable salt thereof

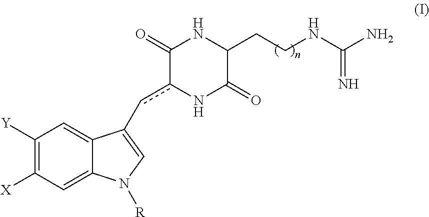

wherein

X and Y are independently selected from the group consisting of hydrogen and a halogen,

[symbol] denotes a single bond or a double bond,

R is selected from the group consisting of hydrogen and C₁-C₆ alkyl, and

n is an integer selected from 1, 2, 3 and 4;

to said mammal. Preferably said mammal is a human.

Another aspect of the present invention is the compound of formula (I) or any pharmaceutically acceptable salt thereof for use in a preservation and/or washing solution for organs. Preferably the solution is for human organs. The organs may be any body part or internal organ but may preferably be selected from the group consisting of hearts, livers, kidneys, and pancreases. Preferably, the organ to be transplanted is immersed and/or washed in said solution.

Yet another aspect is the use of the compound of formula (I) or any pharmaceutically acceptable salt thereof in a solution for the preservation and/or washing of organs. Preferably the solution is for human organs. The organs may be any body part or internal organ but may preferably be selected from the group consisting of hearts, livers, kidneys, and pancreases. Preferably, the organ to be transplanted is immersed and/or washed in said solution.

Another aspect of the present invention is a pharmaceutical preparation comprising the compound of formula (I) or any pharmaceutically acceptable salt thereof and a pharmaceutically acceptable carrier.

Yet another aspect of the present invention is a preservation and/or washing solution for organs comprising the compound of formula (I) or any pharmaceutically acceptable salt thereof and preferably also a pharmaceutically acceptable carrier. Preferably the preservation solution is for human organs. The organs may be any body part or internal organ but may preferably be selected from the group consisting of hearts, livers, kidneys, and pancreases. Preferably, the organ to be transplanted is immersed and/or washed in said solution.

The aging of the human or animal body is closely related to the oxidative stress that said body is subjected to during its lifetime. Since the compound of formula (I) has shown potential in the treatment of diseases related to oxidative stress, it follows that the compound also harbours a potential for the treatment of various signs of aging, including e.g. aging of the skin. Thus, another aspect of the present invention is the use of a compound of formula (I), including salts thereof, for the cosmetic treatment of skin aging. Skin aging may include wrinkles.

Finally, since the compound of formula (I) has shown anti-oxidative potential and since antioxidants may be used in food or feed products, the compounds of the present invention may also be useful as food or feed additives. Therefore yet another aspect of the present invention is the use of a compound of formula (I) as a food and/or feed additive.

It should be noted that embodiments and features described in the context of one of the aspects of the present invention also apply to the other aspects of the invention.

All patent and non-patent references cited in the present application, are hereby incorporated by reference in their entirety.

The invention will now be described in further details in the following non-limiting examples.

### EXAMPLES

### Example 1

### Purification, Isolation and Identification of Barettin

The sponge was collected 08.08.2005 by bottom trawling at 390 m depth in the Barents Sea.

The lyophilized material was extracted twice with ultrapure water (2×1000 ml) at 4° C. in darkness for 8 h. The sample was centrifuged at 4500 g and 5° C. for 30 min, and the pellet was freeze dried. The lyophilized pellet was extracted twice with 1000 ml dichlorometane:methanol (1:1, vol:vol) at 4° C. in darkness for 8 h, and the sample was subsequently filtered through a Whatman No3 filter. The filtrate was reduced to an orange oily liquid at 40° C. and reduced pressure in a rotary evaporator giving 9.17 g organic extract. The organic extract was chromatographed on HP20-resin using a solvent step-gradient system with 5, 25, and 75% aqueous methanol and two last steps of 100% methanol and 100% acetone. The fraction eluting with 50% methanol was reduced to dryness, dissolved in 1 ml 50% aqueous acetonitrile, and barettin was isolated using a Waters HPLC autopurification system. Barettin was isolated on a Waters XTerra C18 column (10×300 mm, 10 μm) with a gradient from 25 to 35% acetonitrile and water, both containing 0.1% formic acid and at a flow rate of 6 ml/min. Two isomers of barettin eluted in two peaks giving 12.9 and 3.9 mg pure compound (retention time 5.1 and 6.3, respectively). High-resolution ESIMS gave m/z 419.0830 [M+H]+, calculated m/z 419.0826 for C₁₇H₁₉BrN₆O₂ ([M+H]+).

### Example 2

### Synthesis of Barettin and De-Bromo Barettin

Barettin and its de-brominated analogue may also be obtained by synthetic methods as provided by the following synthetic steps:

### 2-(benzyloxycarbonylamino)-2-hydroxyacetic acid (3) (Batch 658,661)

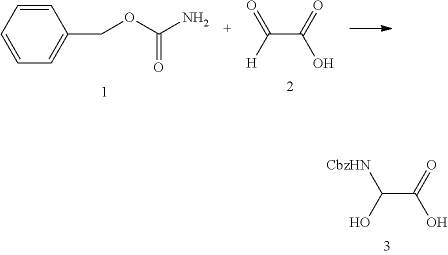 [SMILES: NC(=O)OCc1ccccc1 ; O=CC(=O)O ; O=C(NC(O)C(=O)O)OCc1ccccc1]

A benzyl carbamate (151.17 g/mol, 23 g, 0.15 mol, 1 eq) and glyoxilic acid mono-hydrate (92.05 g/mol, 15.3 g, 0.166 mol, 1.09 eq) were suspended in 125 ml Et₂O. After 1 h stirring at r.t. was obtained the clear mixture. The stirring was continued for o.n. at r.t. Obtained white precipitations were filtered, washed with cold Et₂O and dried over vacuum. The compound (21.23 g, 63%, 13.67 g, 68%) was used directly in the next step without future purification.

¹HNMR (MeOD): ppm, 7.37-7.28 (m, 5H), 5.41 (s, 1H, NH), 5.11 (s, 1H), 4.89 (s, 2H); MS 248.0530 C10H11O5NNa

### Methyl 2-(benzyloxycarbonylamino)-2-methoxyacetate (4) (Batch 659, 662)

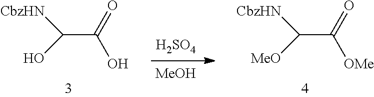

To a solution of Cbz-amino hydroxyacetic acid (see above) in 214 ml of MeOH was added conc. H₂SO₄ (2.1 ml) at r.t. The mixture was stirred for 2 days at r.t. The solvent was evaporated, residue dissolved in EtOAc (˜100 ml), added sat. NaHCO₃ (˜100 ml) at 0° C. (ice bath), the organic layer was washed with H₂O (2×60 ml) and brine (60 ml), dried over MgSO₄. After evaporated solvent it was obtained as a white solid 6.50 g (41%).

### Methyl 2-(benzyloxycarbonylamino)-2-(diethoxyphosphoryl)acetate (5)

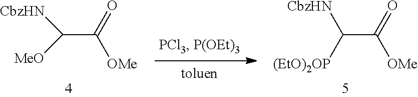

PCl₃ (137.33 g/mol, 1.57 g/ml, R=Et 1.18 ml, 13.3 mmol) was added to a solution of CbzNH-ester (253.24 g/mol, R=Et 3.34 g, 13.3 mmol) in 40 ml toluene at 80° C. The mixture was stirred o.n. at 75-80° C., than added P(OE)₃ (166.16 g/mol, 0.955 g/ml, 23 ml, 13.3 mmol) and continued heating for next 3-6 h. The solvent was evaporated and residue diluted in EtOAc, washed with saturated NaHCO₃ (aqueous NaHCO₃ was extra extracted with EtOAc) and brine. The solvent phase was dried over MgSO₄. Obtained crystals were re-crystallized from EtOAc:C5. White crystals, R=Et, 4.10 g (86%).

¹HNMR (meod): ppm, 7.37-7.29 (m, 5H, Ph), 5.14-5.07 (q, 2H), 4.95-4.89 (dd, 1H), 4.17-4.12 (m, 4H), 3.78 (s, 3H, OCH3), 1.31-1.25 (m, 6H). ¹HNMR (cdcl3): ppm, 7.30-7.25 (m, 5H, Ph), 5.86-5.84 (d, 1H), 5.12-4.89 (q, 2H), 4.89-4.81 (dd, 1H), 4.14-4.07 (m, 4H), 3.78 (s, 3H, OCH3), 1.30-1.22 (m, 6H).

| Batch no | Yield, (g, %) | NMR |
|---|---|---|
| 410 | 3.95, 83% | ¹H |
| 411 | 4.10, 86% | ¹H |
| 656 | — | No prod |
| 660 | 4.05 g/47% | ¹H |
| 663 | 4.025 g/36% | ¹H |

### Methyl 2-amino-2-(diethoxyphosphoryl)acetate (6)

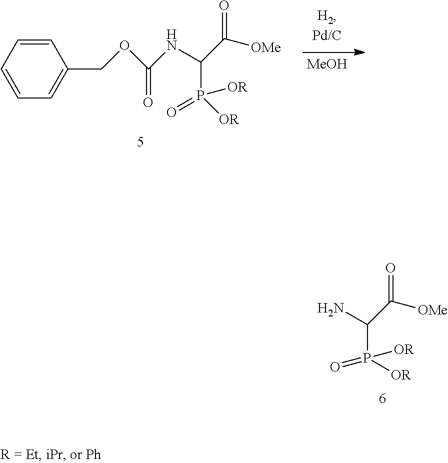

The compound 5 (1 mol) was hydrogenated/debenzylated to the product 6 in the presence of palladium catalyst 10% Pd/C (0.1 mol) and flow of H₂ from balloon in MeOH at r.t. After 1 h or overnight the catalyst was filtered off on celite, washed with MeOH, the solvent evaporated and obtained oil was immediately dissolved in DCM and use to the next step.

TLC: EtOAc or EtOAc:C5 (2:1).

Batch 664: ¹HNMR (CDCl₃, ppm): 4.22-4.15 (m, 4H), 3.98-3.89 (m, 1H, CH), 3.80 (s, 3H), 1.79-1.74 (m, dd, 2H, NH), 1.36-1.32 (m, 6H).

| | R | g, % | time | NMR | info |
|---|---|---|---|---|---|
| 375/376/377/386 | Et | nd | o.n. | — | — |
| 412 | Et | 0.84 g, >100% | 2 h | ¹H | s.m.:prod 1:3 |
| 414 | Et | 0.34 g, 86% | 4 h | ¹H | ok |
| 421 | Et | 0.25 g, 74% | 1.5 h | ¹H | ok |
| 422 | Et | 0.38 g, 85% | 1.5 h | ¹H | ok |
| 423 | Et | 0.31 g, 70% | 1.5 h | — | |
| 429/ | Et | 0.46 g, 90% | 1.5 h | ¹H | ok |
| 430/ | | 0.44 g, 88% | | ¹H | |
| 431 | | 0.42 g, 86% | | — | |
| 432 | Et | 0.49 g, 94% | 2 h | — | |
| 664a | Et | 0.87 g/99% | 2 h | ¹H | ok |
| 664b | Et | 1.67 g/101% | 3 h | ¹H | ok |

### Methyl-2-(N-Boc-Nω,Nω′-bis(Boc)-L-arginyl)amino-2-diethoxyphosphinylacetate (7)

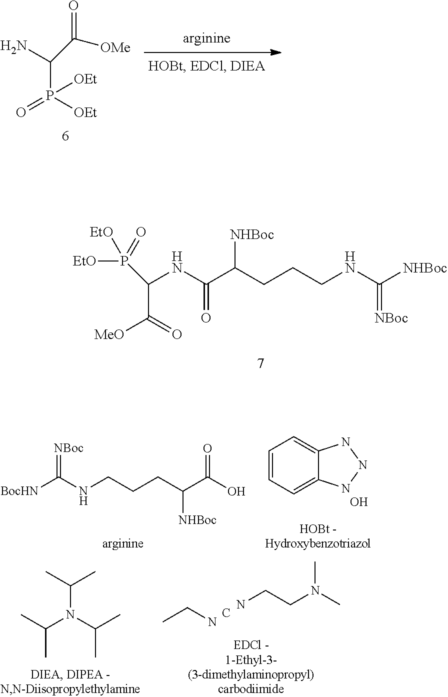

According to Johnson et al., Tetrahedron 2004, (60), 4, pages 961-965. A freshly obtained compound 6 (see above) dissolved in 50 ml DCM was added to an ice cold mixture of Boc-Arg(Boc)₂-OH (4.6 g, 9.7 mmol, 474.56 g/mol), HOBt (1.5 g, mmol, 135.13 g/mol), EDCI (2.6 g, 13 mmol, 191.7 g/mol) and DIEA (2.34 g, mmol, 129.25 g/mol, 0.742 g/ml) in 25 ml of DCM. The reaction mixture was warmed up to r.t. and stirred for 3 d. The solvent was evaporated and added EtOAc and washed with 2×H₂O and brine. The organic phase was dried over MgSO₄. The product was purified on silica EtOAc:pentane (1:1 to 3:1). Obtained white crystals, 1.1 g (12.5%, calc. from comp.5).

665: ¹HNMR (CDCl₃, ppm): 9.48-9.18 (d, 2H), 7.23-7.18 (?), 7.17-7.05 (?), 5.84-5.64 (d, 1H), 5.21-5.11 (m, 1H), 4.18-4.10 (m, 4H), 3.91-3.88 (m, 1H), 3.82-3.79 (d, 3H, OCH3), 1.82-1.76 (m, 1H), 1.69-1.67 (m, 2H), 1.51-1.45 (m, Boc), 1.32-1.29 (m, 4H), 1.27-1.23 (m, 1H). HRMS (MeOH): (M+H)⁺ Calcd for C₂₈H₅₃N₅O₁₂P 682.3429. found: 682.3417.

| | g, % | time | info |
|---|---|---|---|
| 376 | 0.44 g, 20% | o.n. | — |
| 377 | 0.25 g, 25% | o.n. | — |
| 386 | 1.1 g, 12.5% | o.n. | MS 682 ok |
| 424, 425, 426 | 1.92 g, 68% | o.n. | ¹H, t = 6-24 |
| 433, 434, 435 | 2.66 g, 67% | o.n. | ¹H, t = 7-29 |
| 665a | 1.534 g/57% | o.n. | ¹H, MS |
| 665b | 3.43 g/70% | 24 h | ¹H |

### Methyl (2S)-2-(N-Boc-Nω,Nω′-bis(Boc)-L-arginyl)amino-3-(1-Boc-indol-3-yl)prop-2-enoate (8)

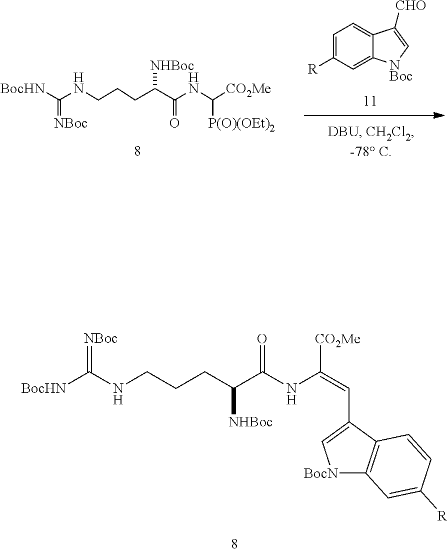

According to Johnson et al., Tetrahedron 2004, (60), 4, pages 961-965 Compound 7 (1.1 g, 1.6 mmol, 681 g/mol, 1 eq) was dissolved in 8 ml DCM (dried over molecular sieves) under argon. To the solution was slowly added DBU (1,8-diazabicyclo[5.4.0]undec7en, 0.5 ml, 3.2 mmol, 152.24 g/mol, 1.018 g/mol) in 8 ml DCM at −78° C. (dry ice/acetone). The mixture was stirred for 30 min at −78° C., and added 6-bromo-N-(tert-butoxycarbonyl)-indole-3-carboxyaldehyde 11 (0.52 g, 1.6 mmol, 323 g/mol) in 8 ml DCM. The reaction mixture was allowed warm up to r.t. and stirred o.n. The solvent was evaporated, added EtOAc, washed with H₂O and brine, organic phase was dried over MgSO₄, and the product was purified on silica (6% MeOH:DCM or EtOAc:C5 1:2). Obtained white solid (1.06 g, 78%).

¹HNMR (400 MHz, CDCl₃, ppm): δ 9.47 (s, 1H), 9.34 (s, 1H), 8.42 (s, 1H), 8.33 (s, 1H), 7.82 (s, 1H), 7.74 (s, 1H), 7.56 (d, J=8.4 Hz, 1H), 7.39 (dd, J=8.5, 1.8 Hz, 1H), 6.16 (s, 1H), 4.56-4.46 (m, 1H), 4.09 (s, 1H), 3.78 (s, 3H), 3.49 (d, J=2.6 Hz, 1H), 1.89-1.81 (m, 2H), 1.78-1.74 (m, 2H), 1.66 (s, 9H), 1.59 (d, J=2.3 Hz, 2H), 1.52 (s, 9H), 1.46 (s, 9H), 1.35 (s, 9H). HRMS (MeOH): Calcd for C₃₈H₅₆N₆O₁₁Br 851.3193. found: 851.3168.

| | | | Yield | | |
|---|---|---|---|---|---|
| | R-indol | base | (g, %) | time | info |
| 382a | H— | DBU | 0.38 g, 97% | o.n. | MS |
| 382b | H— | TGN | 0.2 g, 51% | o.n. | — |
| 388 | Br— | DBU | 1.06 g, 78% | 2 days | MS, t = 6-9 |
| 438a | —/— | —/— | 0.72 g, 90% | 2 days | ¹H, t = 6-12 |
| 438b | —/— | —/— | 1.61 g, 80% | —/— | ¹H, t = 5-10 |
| 443/444 | —/— | —/— | 1.76 g | —/— | ¹H, MS, |
| 666a | —/— | —/— | 1.75 g/94% | —/— | ¹H, MS, |
| 666b | —/— | —/— | 3.56 g/87% | | ¹H, MS, |

Barettin (R═Br) (9) (designated MBC-011)

### a. Procedure According to Johnson et al., Tetrahedron 2004, (60), 4, 961-965

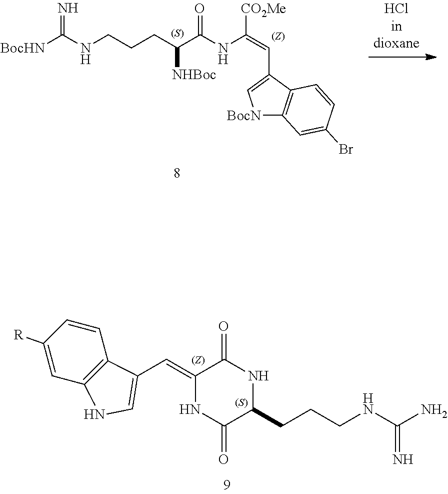

Procedure for preparation of barettin: TFA (trifluoro acetic acid, 0.86 ml, 11 mmol, 114.02 g/mol, 1.489 g/ml) was added to the solution of compound 8 (0.52 g, 0.61 mmol, 851 g/mol) in 10.4 ml DCM, and the mixture was stirred o.n. at r.t. (Check MS during the reaction). The solvent was evaporated, to the red oil was added 0.75 ml of acetic acid in 10 ml of 1-butanol and 60 μNMM (N-methylmorpholine). The mixture was refluxed (128° C.) for 2-4 h, after cooled to r.t., washed with H₂O and brine, the organic phase was dried over MgSO₄, evaporated solvent, obtained white with reddish solids 0.22 g (86%) was dried over vacuum.

| | 9 as TFA | | |
|---|---|---|---|
| R = Br | salts/9 after hplc/yield | time | info |
| 384 | 0.23 g/—/— | — | MS, ¹H |
| 389 | 0.22 g/—/— | o.n., 4.5 h | MS, ¹H |
| 437 | 1.14 g/0.043 g/34% | o.n, 3.5 h | MS |
| 441 | 0.4 g/0.145 g/41% | o.n., 4 h | MS |
| 442 | 0.75 g/0.354 g/45% | o.n., 2 h | MS |
| 445 | 0.9 g/0.251 g/29% | o.w., 3 h | MS |

Debromo-barettin (R═H) was prepared accordingly, i.e. following a procedure analogous to the above with R═H.

### b. Alternative Procedure

### Methyl 2-(2-amino-5-guanidinopentanamido)-3-(6-bromo-1H-indol-3-yl)acrylate (12)

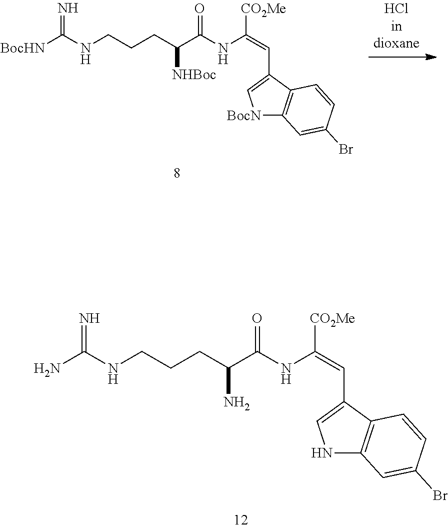 [SMILES: COC(=O)/C(=C/c1cn(C(=O)OC(C)(C)C)c2cc(Br)ccc12)NC(=O)[C@H](CCCNC(=N)NC(=O)OC(C)(C)C)NC(=O)OC(C)(C)C ; COC(=O)/C(=C/c1c[nH]c2cc(Br)ccc12)NC(=O)[C@@H](N)CCCNC(=N)N]

4M HCl in dioxane (3.8 ml) was added to compound 8 (171 mg, 0.2 mmol). The mixture was stirred o.n. at r.t. Obtained reddish precipitations were filtered and dried over vacuum. Yield over 100% (consist HCl-salts).

¹HNMR (400 MHz, DMSO-d6) δ 12.16 (s, 1H), 10.15 (s, 1H), 8.46 (s, 3H), 8.20 (d, J=2.7 Hz, 1H), 7.87 (s, 1H), 7.77-7.69 (m, 2H), 7.66 (d, J=1.8 Hz, 1H), 7.26 (dd, J=8.4, 1.8 Hz, 1H), 4.58 (s, 2H), 4.18 (s, 1H), 3.72 (s, 3H), 3.22 (q, J=6.8 Hz, 2H), 1.91 (s, 2H), 1.72 (s, 2H). HRMS (MeOH): Calcd for C₁₈H₂₄BrN₆O₃ 451.1093. found: 451.1089.

Barettin (9) (from (12))

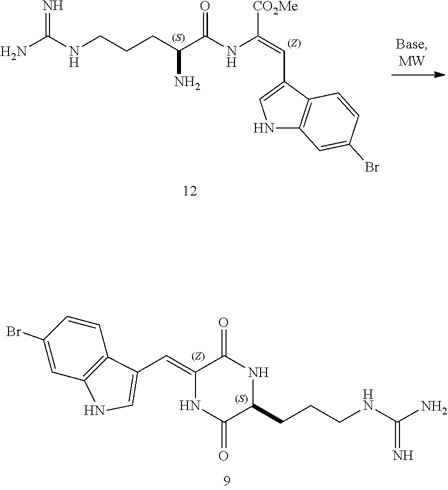

Crude compound 12 (˜0.13 g) was dissolved in 12 ml EtOH and added base. The mixture was irradiated in microwave at 80° C. for 1-2 h. The solvent was evaporated, reddish residue was dissolved in n-BuOH and washed with 3×20 ml H₂O. The organic layer was dried over Na₂SO₄. Obtained red solid.

HRMS (MeOH): Calcd for C₁₇H₂₀BrN₆O₂ 418.0753. found: 419.0821.

| | Yield from (8) after | | |
|---|---|---|---|
| | Hplc | base | info |
| 668 | — | NMM | MS, ¹H, ok |
| 670a | 57 mg/— | Et₃N | MS |
| 670b | 28 mg/39% | —/— | MS |
| 673 | 24 mg/19% | Cs₂CO₃ | MS |

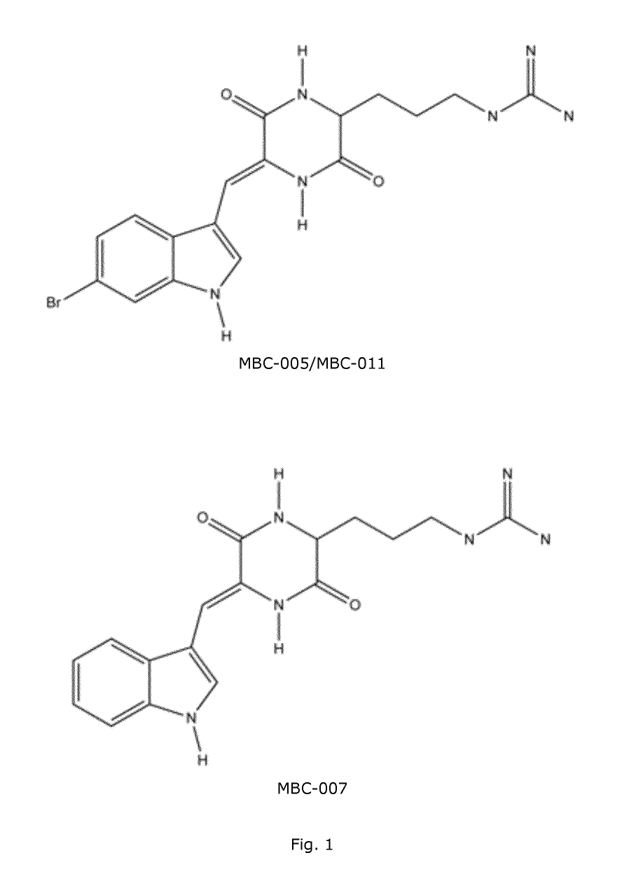

In the examples and figures the main isomer of isolated barettin is designated MBC-005 while the de-brominated synthetic analogue, i.e. the Z isomer of barettin wherein bromine is replaced by hydrogen, is designated MBC-007, unless otherwise stated. The compound designated MBC-011 corresponds to MBC-005 but was provided by the above synthetic method, and is thus the Z-isomer. It is also the (S)-enantiomer, as provided by using the (Boc protected) natural amino acid arginine as starting material. The compound structures are also shown in FIG. 1.

Anti-Oxidative Effects

The inventors have demonstrated anti-oxidative effects of barettin and analogues as outlined in the below examples. Since oxidative stress is related to a number of diseases, including particularly cardiovascular disease and cancer, these results support a novel therapeutic potential of barettin towards such diseases.

### Example 3

### FRAP Assay

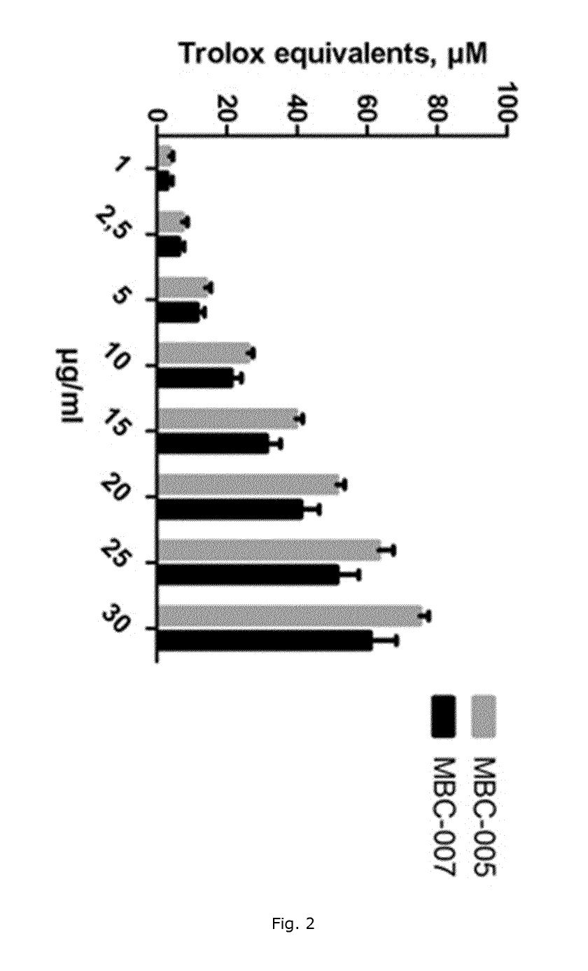

The inventors used the biochemical assay FRAP (ferric reducing antioxidant power) to obtain an indication of the antioxidant potential of MBC-005 (isolated brominated) and MBC-007 (synthetic de-brominated). A dose-response activity was observed for both compounds (FIG. 2). At a concentration of 30 μg/ml (71.6 μM) MBC-005 had a FRAP value of 77 μM TE. For comparison, 30 μg/ml (105 μM) luteolin had a FRAP value of 151 μM TE (data not shown).

Reagents were prepared according to benzie, I.F.F. et al. Analytical Biochemistry 1996, 239, 70-76 and carried out in a DTX 880 Multimode Detector (Beckman Coulter, Calif., USA) at 595 nm. Trolox (Sigma, 238813) was used to prepare the standard curve (0-250 μM; working concentration). The FRAP reagent (TPTZ (2,4,6-Tris(2-pyridyl)-s-triazine): Fluka, 93285; Fe: Merck 103943) was prepared daily. The assay was carried out in clear 96-well plates, with 20 μl sample and 150 μl FRAP-reagent added to each well in duplicates. Water was used as a blank. Samples were incubated at 37° C. for 30 min before reading the plate. The blank was subtracted from each sample and a standard curve was created from the average absorbance of the duplicated Trolox samples. The equation generated from the standard curve was used to calculate the trolox equivalents (TE) from each sample. Results were expressed as μM TE.

### Example 4

### ORAC Assay

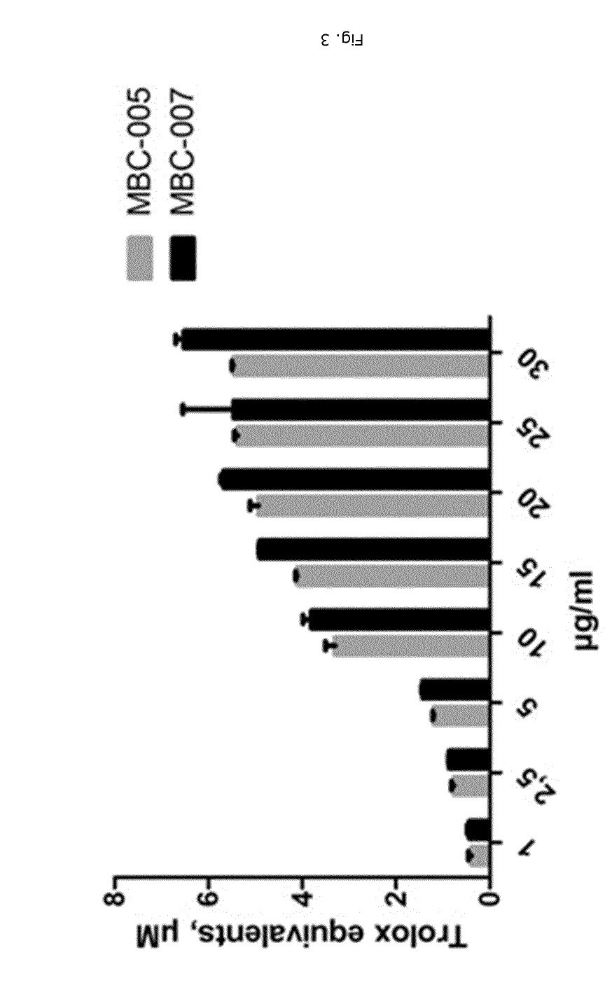

The inventors used the biochemical assay ORAC (oxygen radical absorbance capacity) to obtain an indication of the antioxidant potential of MBC-005 (isolated brominated) and MBC-007 (synthetic de-brominated). A dose-response activity was observed for both compounds (FIG. 3). At a concentration of 30 μg/ml (71.6 μM) MBC-005 had a 5.5 μM TE.

The method has been modified from Huang, D. et al., Journal of Agricultural and Food Chemistry 2002, 50, 4437-4444. The assay was carried out in black 96-well plates (Nunc 7350004) and using a Victor3 Plate Reader (Perkin Elmer, Mass., USA) at 37° C. (exitation 486 nm, emission 520 nm). All reagents were dissolved in 75 mM phosphate buffer (PB) (pH 7,4). Different concentrations of the MBC-005 and MBC-007 preparations were added in duplicates followed by addition of 125 μl fluorescein (Fluka, 46960) (52 nM final concentration). After a 15 minute incubation at 37° C., 60 μl AAPH (2,2′-Azobis(2-methylpropionamidine) dihydrochloride; Sigma-Aldrich, 440914) was quickly added to each sample (44 mM final concentration). Fluorescence was recorded 25 times at 37° C. with a 70 sec delay between repeats. Trolox (0-25 μM working concentration) was included in each run to make a standard curve. PB was used as a blank and for the 0 μM Trolox sample. Area under the curve (AUC) was calculated by subtracting the AUC_{Blank} values. A standard curve was created using the Trolox values and Trolox equivalents of the samples were calculated from the resulting equation. Results were expressed as μM TE.

### Example 5

### CLPAA Assay

HepG2 cells were grown in MEM Earle's medium (F0325) supplemented with gentamycin (10 μg/ml, A2712), non-essential amino acids (1%, K0293), sodium pyruvate (1 mM, L0473), L-alanyl-L-glutamine (2 mM, K0302) and fetal bovine serum (10%, S0115) and incubated at 37° C. with 5% CO₂. Media and supplements were from Biochrom (Berlin, Germany).

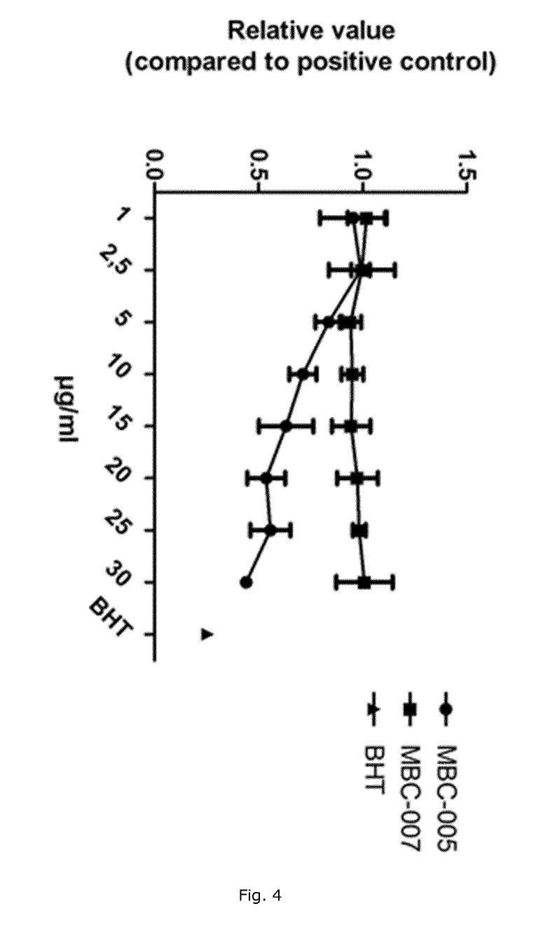

The inventors used the cellular lipid peroxidation antioxidant activity (CLPAA) assay. This assays measures the intracellular ROS (reactive oxygen species) and membrane antioxidant activity, respectively. The brominated barettin molecule gave a 55% reduction in oxidation compared to the control in the CLPAA assay. The de-brominated synthetic analogue however did not show any significant activity (FIG. 4) in this assay.

Approximately 80.000 HepG2 cells per well were seeded in black 96 well plates with clear bottom (#3603, Corning, N.Y., USA) and incubated overnight. The cells were labelled with C11-BODIPY (#D3861, Invitrogen, Eugene, Oreg.) for 30 min and incubated for 1 h with various concentrations of the test compounds. Cumene hydroperoxide (cumOOH, #247502, Sigma-Aldrich, St. Louis, Mo.) was added to initiate lipid peroxidation and the plate was immediately placed in a Victor3 Plate Reader (Perkin Elmer, Mass., USA). Both red (590/7 nm (exitation), 632/45 nm (emission)) and green (485/14 nm, 520/10 nm) fluorescence was recorded during ˜1 h. Cells were washed with PBS between additions of new reagents. Total reaction volume was 100 μl. All incubations were carried out at 37° C. with 5% CO₂. Percent inhibition was calculated relative to the positive control (cumOOH without test compound).

### Example 6a

### Inhibition of Cytokines Related to Inflammation

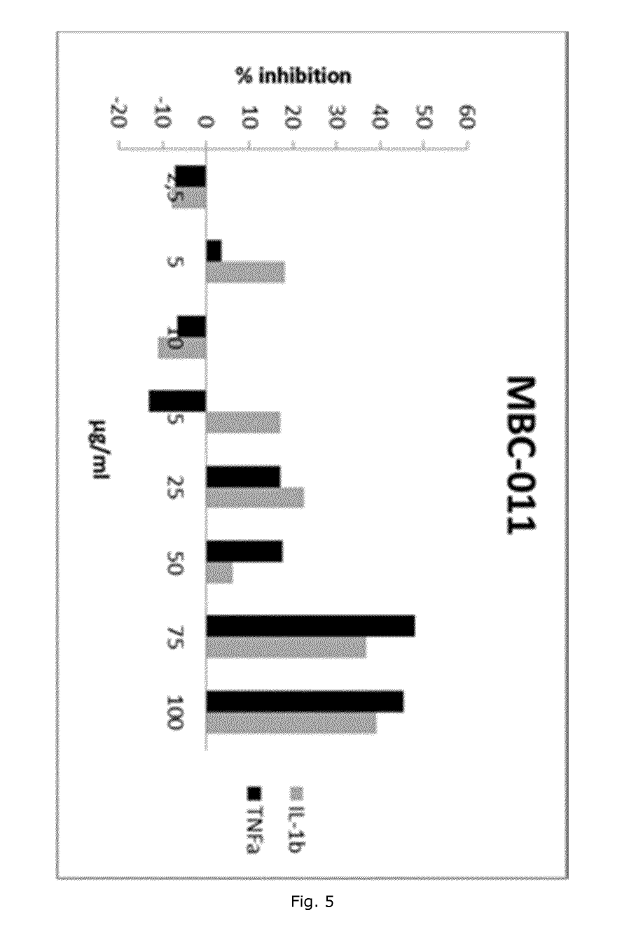

Tumor necrosis factor-alpha (TNF-α) and Interleukin-1 beta (IL-1β) are both cytokines (small cell-signalling protein molecules) linked to inflammatory responses. Thus, the present inventors have tested the ability of isolated barettin (designated MCB-011 in the present experiment) to inhibit the secretion of these cytokines in LPS induces monocytes (THP-1 cells). The results are shown in FIG. 5, where barettin displays significant inhibition of TNF-α and IL-1β at concentrations of 75 and 100 μg/ml.

THP-1 cells were grown in RPMI-1640 supplemented with gentamycin and FBS and incubated at 37° C. with 5% CO₂. To differentiate the monocytes into macrophages, 50 ng/ml PMA was added. Approximately 10⁵ cells supplemented with 50 ng/ml PMA were seeded in 96 well plates and incubated at 37° C., 5% CO₂ for 48 hours. The cells were controlled using a microscope after 24 h to make sure they had started to differentiate. After 48 h the cells were washed and new RPMI (w/o PMA) added before another 24 h incubation.

The cells were added 90 μl fresh medium and 10 μl test compound at various concentrations in duplex. After incubation for 1 h, 1 ng/ml LPS was added to all samples except negative controls. Incubation for 6 h at 37° C. followed. The reactions were stopped by freezing the plates at −80° C. immediately after incubation. One day prior to ELISA testing MaxiSorp 96F-well plates were coated with 2 μg/ml capture antibody (Ab) and placed in the refrigerator overnight. Between every step the plates were washed with TBS (pH 7,4) added 0.05% Tween 20. A volume of 200 μl blocking buffer (TBS added 2% BSA) was added to the plates and they were incubated for 1 h with shaking at room temperature. Samples were diluted appropriately and added to the plates. A standard was added to each plate. Further incubation at room temperature for 2 h with shaking was performed. Biotin anti-human Ab was diluted in assay diluent (TBS with 1% BSA) to 3 μg/ml and added to each well. 1 h incubation at room temperature followed. Diluted ExtrAvidin®-Alkaline Phosphatase was added and plates incubated for 30 min. pNPP substrate was dissolved 1 M diethanolamin buffer pH 9.8 to 1 mg/ml, 100 μl added to each well and the plates incubated for 45 min. Results were read at DTX880 at 405 nm.

### Example 6b

### Inhibition of Cytokines Related to Inflammation

Monocytes are well-recognised to respond to inflammatory stimuli by releasing a range of cytokines; IL-6 is a highly expressed pro-inflammatory cytokine in activated monocytes. The principle of this assay was to compare the effect of pre-incubating human cells from a monocyte-derived cell line (THP-1) with a known anti-inflammatory drug (dexamethasone; Dex), or a range of concentrations of barettin, on IL-6 generation in response to the inflammatory stimulus, lipopolysaccharide (LPS).

THP-1 cells (Health Protection Agency, Porton Down, UK) were maintained at 37° C. with 5% CO₂ in RPMI 1640 containing 10% foetal bovine serum, 2 mM L-glutamine with 100 IU/ml penicillin and 100 μg/ml streptomycin (PAA laboratories Ltd, Yeovil, UK). The cells were grown in suspension and then plated out in 24-well plates at 2×10⁵ cells per well.

Treatments:
a. vehicle control (RPMI 1640+0.5% DMSO)
b. 10 μM Dex for 24 h
c. 10 μg/ml LPS for 24 h
d. Pre-treated with 10 μM Dex for 30 min followed by 10 μg/ml lipopolysaccharide LPS for 24 h
e. Pre-treated with barettin at 1, 3, 10, 30 and 100 μg/ml for 30 min followed by 10 μg/ml LPS for 24 h

The concentration of Dex and barettin remained consistent throughout the incubation period; the final DMSO concentration was 0.5%. Cells were incubated at 37° C. and 5% CO₂ for 24 hours.

Following incubations, cells and supernatants were harvested into 1.5 ml Eppendorf® tubes and supernatants were cleared of cells and debris by centrifugation at 300 g for 3 min at room temperature. Supernatants were transferred to clean 1.5 ml Eppendorf® tubes and stored at −80° C.

IL-6 expression was measured by ELISA (eBioscience, human IL-6 Platinum ELISA) according to the manufacturer's instructions. Data were collected using a VarioSkan Flash plate reader and Skanit software v2.4.3 (Thermo Fisher Scientific, Basingstoke, UK).

Pilot data showed that treatment of THP-1 cells with the barettin alone did not result in the up-regulation of IL-6 expression.

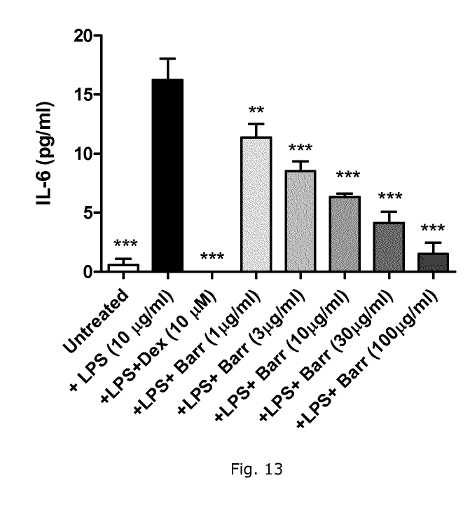

The results are shown in FIG. 13. There is a clear concentration-dependent effect of barettin (1-100 μg/ml) on LPS-induced generation of IL-6. The effect was significant, even at the 1 μg/ml (˜2.4 μN) concentration. Dexamethasone (10 μN) abolished IL-6 generation in LPS-treated cells. Whilst cytotoxic effects of barettin cannot be ruled out at the highest concentration, the results for barettin at 30 μg/ml and below reflect a genuine anti-inflammatory effect in these cells.

Cytotoxicity

Although some cytotoxicity may be tolerated in the treatment of certain diseases, it is generally advantageous if an active species of a medicament displays little or no cytotoxicity at the concentrations at which the efficacy towards the relevant disease becomes significant. In the below examples the inventors have shown that barettin and analogues show little or no cytotoxicity at therapeutically relevant concentrations.

### Example 7

### Cytotoxicity Assay for HepG2 and MRC 5 Cells

The previously reported anti-fouling properties of barettin could indicate toxicity in the HepG2 cells and bromine gives barettin a cytotoxic character. It was therefore important to check the cytotoxicity of the compounds. Hepatocytes are good models for studying toxicity since the liver is the primary site of drug metabolism and biotransformation. Drugs are often bio-transformed in the liver to make the metabolites more hydrophilic and this may render a drug compound toxic by producing toxic metabolites. Hepatocytes and normal lung fibroblasts were used to test the cytotoxicity.

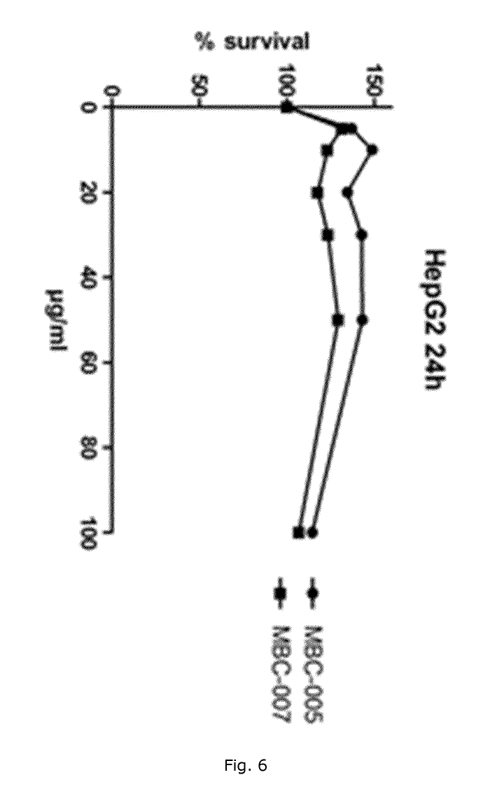

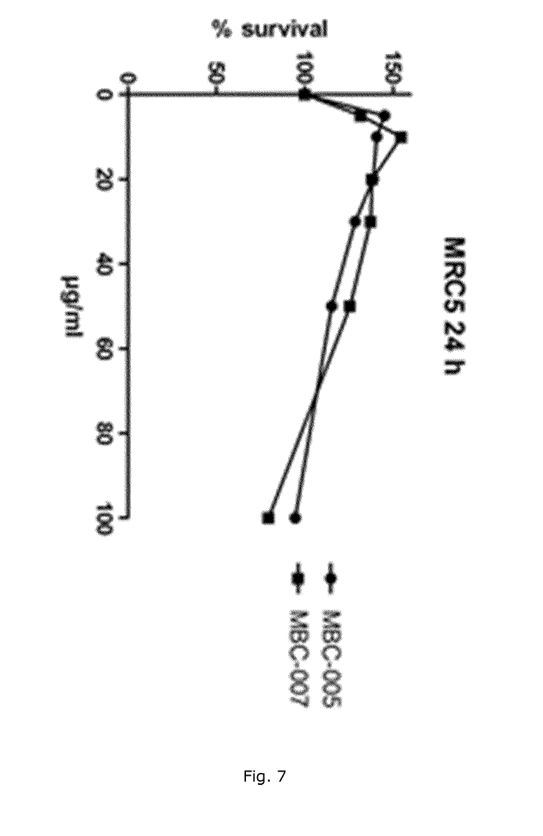

Cytotoxicity was tested on HepG2 and MRC-5 cells (normal lung fibroblasts) for 2 h (HepG2) and 24 h (HepG2 and MRC-5) using the CellTiter 96 AQueous One Solution Assay (Promega). MBC-005 and MBC-007 were not toxic to the HepG2 or the MRC5 cells in the concentrations tested (FIG. 6 and FIG. 7). In the CLPAA assays the cells were exposed to the test compounds for 1 h before washed. Thus a 2 h exposure would detect whether the MBC-compounds are likely to cause cell death and false results in the CLPAA assays. MRC5 and HepG2 cells were also exposed to the barettin compounds for 24 h. This would reveal more long-term damage or whether any toxicity was caused by something other than membrane lysis. The cytotoxicity of isolated barettin was also tested over a period of 72 h (data not shown) and the compound did not show cytotoxicity until concentrations reached 100 μg/ml. This was above the concentrations used in the CLPAA assay.

HepG2 and MRC-5 cells were grown in MEM Earle's medium (F0325) supplemented with gentamycin (10 μg/ml, A2712), non-essential amino acids (1%, K0293), sodium pyruvate (1 mM, L0473), L-alanyl-L-glutamine (2 mM, K0302) and fetal bovine serum (10%, S0115) and incubated at 37° C. with 5% CO₂. Media and supplements were from Biochrom (Berlin, Germany).

Cytotoxicity was studied in HepG2 and MRC-5 cells (normal human lung fibroblasts) for 2 h (HepG2) and 24 h (HepG2 and MRC-5). For the 2 h study, HepG2 cells were seeded per well. For the 24 h study, 50 000 HepG2 cells and 7 500 MRC-5 cells were used. Cells were grown over night, then washed with PBS and added 50 μl test compound at various concentrations diluted in MEM Earle's supplemented as above but without FBS. After incubation, 10 μl of CellTiter 96® AQᵤₑₒᵤₛ One Solution Reagent (Promega, Madison, Wis., USA) was added and plates were then incubated further for 1 h. Absorbance was measured at 485 nm in a DTX 880 Multimode Detector. Results were calculated as % survival compared to a positive control.

### Example 8

### In Vivo Assay

Animals and Housing

Thirty six female apoE^{-/-} mice (C57BL6/J), 5-wk-old, were obtained from Taconic. After one week of acclimation, the mice were ear marked and randomly allotted to three experimental groups with equal number of cages per treatment. All mice were housed in the same room at 21° C. and 55% relative humidity, on a 12-h-day/-night-cycle (light on at 0600 h) in a conventional laboratory animal unit. The mice consumed feed ad libitum for 16 weeks and cages and bedding were changed once per week. The study was approved by the Norwegian National Animal Research Committee and performed following Federation of European Laboratory Animal Science Associations recommendations and according to the Norwegian legislation on the care and use of experimental animals. The mice were pathogen free, stated by a health certificate.

Diets

The mice were allocated to 3 groups (12 animals per group) receiving a Western type diet (WD, control diet) (Research diet D12079B, 17 energy percent protein, energy percent carbohydrates and 40 energy percent fat). The Barettin diet (BAR) was supplemented with 57 mg Barettin per kg diet, and the Olivita diet (OLI) was WD enriched with 1% (wt/wt) of Olivita (mixture of seal oil and olive oil obtained from Olivita AS). The experimental diets were designed to be isocaloric and isocholesterolic.

Analysis of Atherosclerosis

The mice were subjected to 3 hours of fasting before they were euthanized. Blood was drawn by heart puncture and the mouse was perfused through the left ventricle with sterile saline (0.9%) until no residual blood remained in the major organs. The entire aorta from the proximal ascending aorta to the bifurcation of the iliac arteries was cleaned in situ from the periadventitial tissue and dissected from the aortic arch down to the iliac bifurcation. The aorta was opened longitudinally, fixed and prepared mounted on slides. Aorta images were made by scanning the objective, ImageJ software was used to evaluate the lesion area and the extent of atherosclerosis was reported as percentage of the total area of an artery occupied by atherosclerotic lesions.

Serum Cholesterol, LDL Cholesterol, Triacylglycerol and oxLDL Determination

Serum total cholesterol, LDL-cholesterol, and triglyceride concentrations were determined by conventional enzymatic kits using a MaxMat bioanalyzer (MaxMat PL II, Montpellier, France). Serum oxLDL was determined using a ELISA kit from Uscn Life Science Inc. according to the manufacturers' instructions.

Statistical Analysis

The results are presented as means 6 SEM. The Kolmogorov-Smirnov and Shapiro-Wilk tests were used to determine the distribution of the variables, and non-normally distributed variables were log transformed before statistical analysis. Data were analyzed by ANOVA followed by Tukey's post hoc test or the Kruskal-Wallis test (SPSS 17.0; SPSS). P, 0.05 was considered significant.

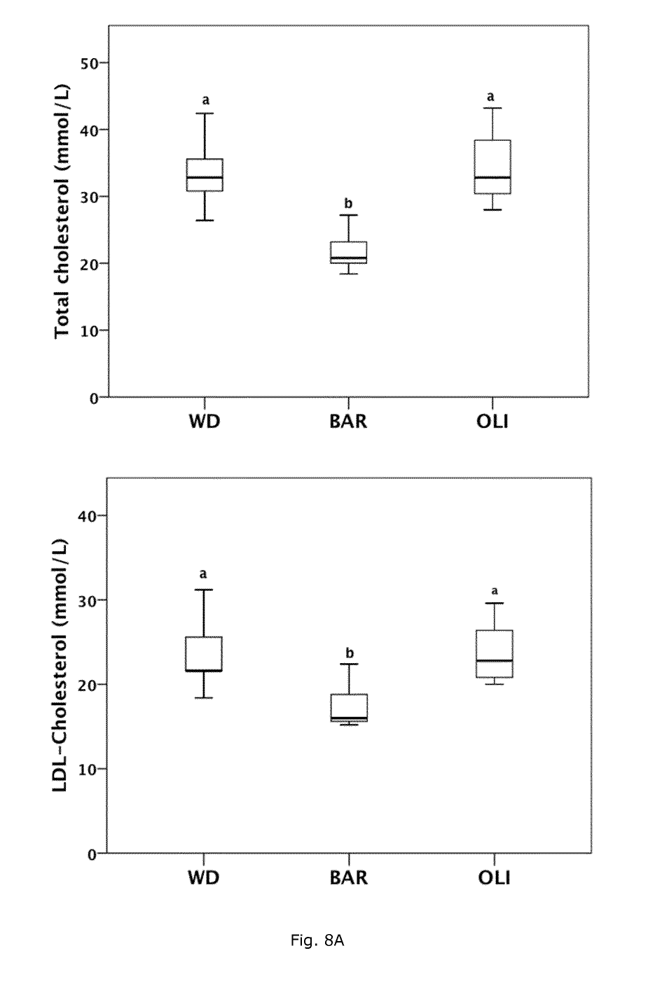

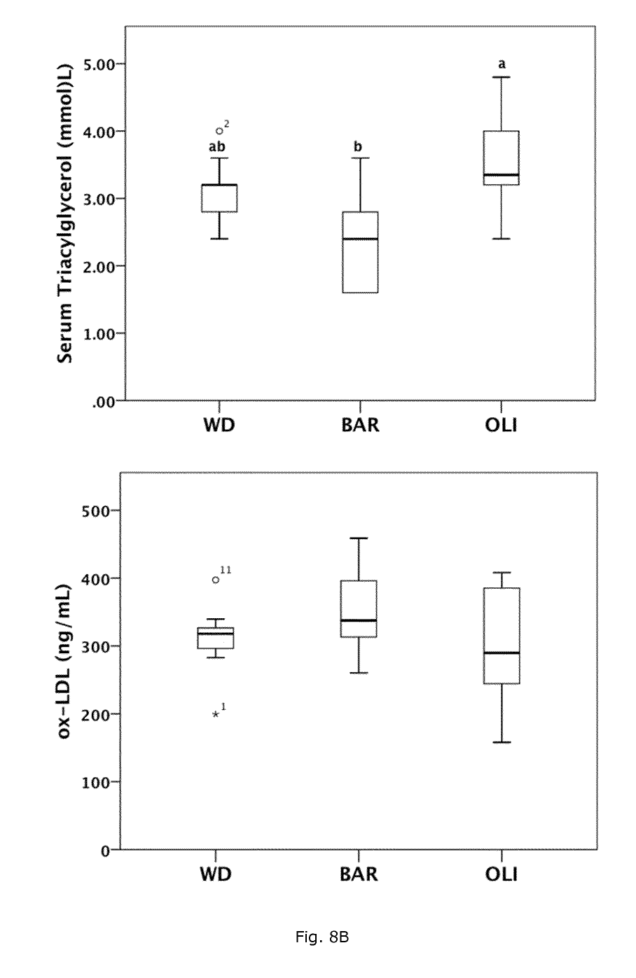

FIGS. 8A, 8B show the effects of 7 weeks feeding of apolipoprotein E-deficient mice with western diet (WD) compared with WD containing barettin (BAR) or Olivita (OLI) on serum total cholesterol (mmol/L) and Serum LDL cholesterol (mmol/L) (FIG. 8A), and triacylglycerides (mmol/L) and serum ox-LDL (ng/mL) (FIG. 8B).

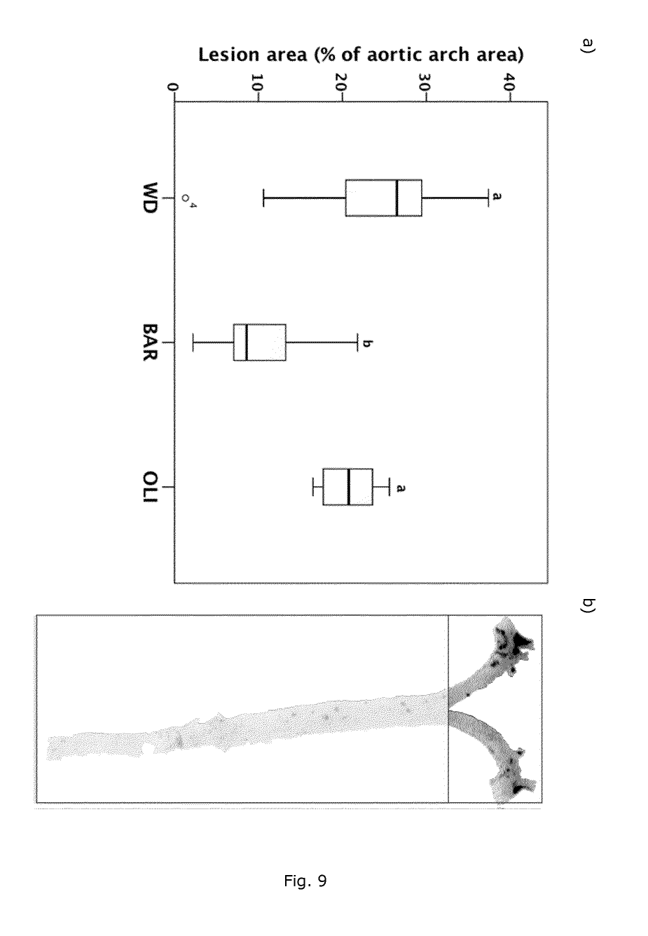

FIG. 9 a) shows the effects of 16 weeks feeding of apolipoprotein E-deficient mice with western diet (WD) compared with WD containing barettin (BAR) or Olivita (OLI) on atherosclerostic lesion development in mice aortas. Depicted here is the lesion area in the aortic arch (relevant aorta area as depicted in b)); Data are medians+/−95% confidence intervals. Labelled boxes without a common letter differ, i.e. a≠b; ANOVA with Games-Howell post hoc test, not assuming equal variances.

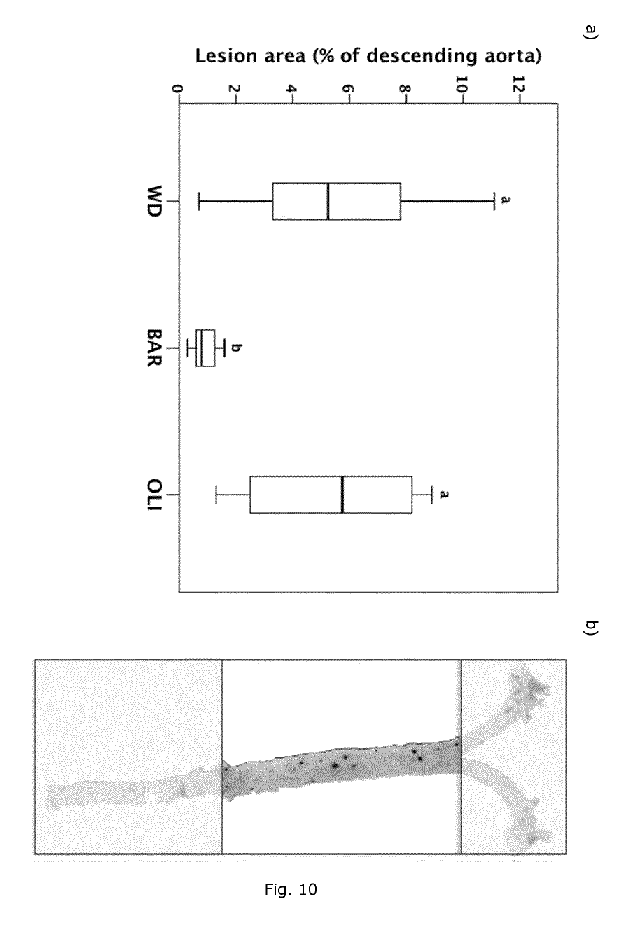

FIG. 10 a) shows the effects of 16 weeks feeding of apolipoprotein E-deficient mice with western diet (WD) compared with WD containing barettin (BAR) or Olivita (OLI) on atherosclerostic lesion development in mice aortas. Depicted here is the lesion area in the descending aorta (relevant aorta area as depicted in b)); Data are medians +/−95% confidence intervals. Labelled boxes without a common letter differ, i.e. a≠b; ANOVA with Games-Howell post hoc test, not assuming equal variances.

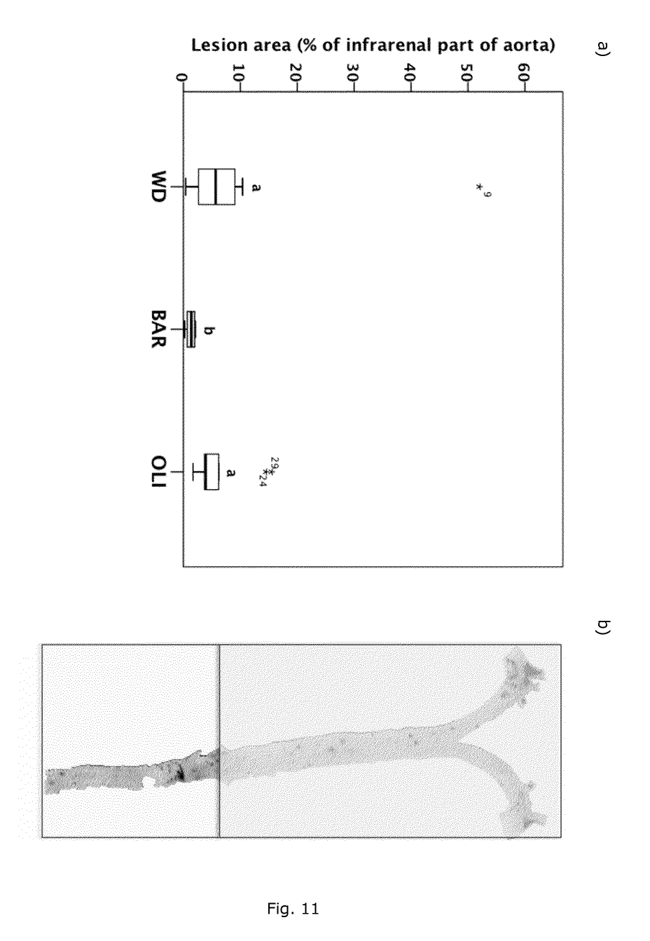

FIG. 11 a) shows the effects of 16 weeks feeding of apolipoprotein E-deficient mice with western diet (WD) compared with WD containing barettin (BAR) or Olivita (OLI) on atherosclerostic lesion development in mice aortas. Depicted here is the lesion area in the infrarenal part of aorta (relevant area as depicted in b)); Data are medians+/−95% confidence intervals. Labelled boxes without a common letter differ, i.e. a≠b; ANOVA with Games-Howell post hoc test, not assuming equal variances.

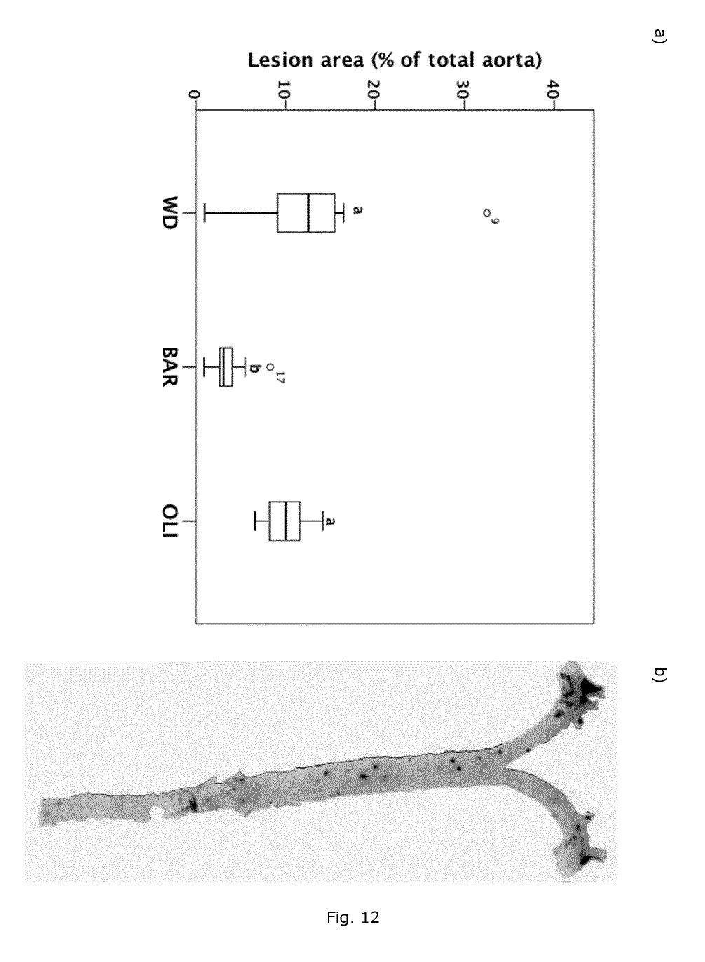

FIG. 12 a) shows the effects of 16 weeks feeding of apolipoprotein E-deficient mice with western diet (WD) compared with WD containing barettin (BAR) or Olivita (OLI) on atherosclerostic lesion development in mice aortas. Depicted here is the lesion area in the total aorta (total aorta depicted in b)). Data are medians+/−95% confidence intervals. Labelled boxes without a common letter differ, i.e. a≠b; p<0.001 ANOVA with Games-Howell post hoc test, not assuming equal variances.

### REFERENCES

Sölter S. et al., tetrahedron letters, 2002, 43, 3385-3386
Johnson, A.-L. et al., tetrahedron 2004, 60, 961-965
WO 03/081199
Hedner, E. et al., J. Nat. Prod. 2006, 69, 1421-1424
Benzie, I. F. F. et al. Analytical Biochemistry 1996, 239, 70-76
Huang, D. et al., Journal of Agricultural and Food Chemistry 2002, 50, 4437-4444.

1. A compound of formula (I) 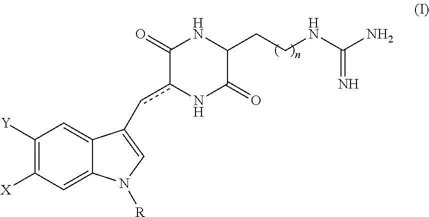
wherein
X and Y are independently selected from the group consisting of hydrogen and a halogen, [symbol]
denotes a single bond or a double bond,
R is selected from the group consisting of hydrogen and C₁-C₆ alkyl, and
n is an integer selected from the group consisting of 1, 2, 3 and 4;
or any pharmaceutically acceptable salt thereof for use in treatment of diseases related to oxidative stress selected from the group consisting of Cancer, Cardiovascular disease, Parkinson's disease, Motor neuron disease, Huntington's disease, Atherosclerosis, Myocardial infarction, Bipolar disorder, Fragile X syndrome, Sickle Cell Disease, Lichen Planus, Vitiligo, Autism, and Chronic Fatigue Syndrome.

2. A compound of formula (I) according to claim 1, wherein said disease is a cardiovascular disease selected from the group consisting of Coronary heart disease, Cardiomyopathy, Hypertensive heart disease, Cor pulmonale, Inflammatory heart disease, Endocarditis, Inflammatory cardiomegaly, Myocarditis, Valvular heart disease, Stroke, cerebrovascular disease, and Peripheral arterial disease.

3. A compound of formula (I) 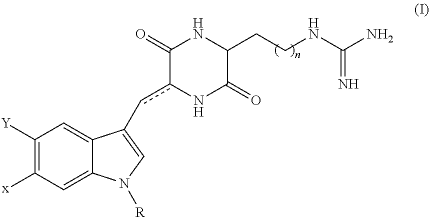
wherein
X and Y are independently selected from the group consisting of hydrogen and a halogen, [symbol]
denotes a single bond or a double bond,
R is selected from the group consisting of hydrogen and C₁-C₆ alkyl, and
n is an integer selected from the group consisting of 1, 2, 3 and 4;
or any pharmaceutically acceptable salt thereof for use in treatment of inflammatory diseases selected from the group consisting of acid reflux/heartburn, acne, Acne vulgaris, allergies and hypersensitivities, ankylosing spondylitis, appendicitis, arthritis, asthma, atherosclerosis, autoimmune diseases, bronchitis, Bursitis, carditis, celiac disease, chronic pain, Chronic prostatitis, Colitis, Crohn's disease, cirrhosis, colitis, cystitis, dermatitis, diabetes, diverticulitis, dry eyes, edema, emphysema, eczema, fibromyalgia, gastroenteritis, gingivitis, Glomerulonephritis, heart disease, hepatitis, high blood pressure, inflammatory cardiovascular disease, Inflammatory bowel diseases, insulin resistance, interstitial cystitis, joint pain, systemic lupus erythematous, meningitis, metabolic syndrome (syndrome X), myositis, nephritis, obesity, osteoarthritis, osteopenia, osteoporosis, Parkinson's disease, periodontal disease, Pelvic inflammatory disease, phlebitis, polyarteritis, polychondritis, psoriasis, psoriatic arthritis, Reperfusion injury, rheumatoid arthritis, Rhinitis, Sarcoidosis, scleroderma, sinusitis, Sjögren's syndrome, spastic colon, systemic candidiasis, tendonitis, tonsillitis, Transplant rejection, vaginitis, ulcerative colitis, and Vasculitis.

4. A compound of formula (I) according to claim 3, wherein said disease is Atherosclerosis.

5. A compound of formula (I) according to claim 1, wherein the treatment is a prophylactic treatment.

6. A compound of formula (I) according to claim 1, wherein X is a halogen and Y is hydrogen.

7. A compound of formula (I) according to claim 1, wherein the halogen is bromine.

8. A compound of formula (I) according to claim 1, wherein R is hydrogen.

9. A compound of formula (I) according to claim 1, wherein n is 2.

10. A compound of formula (I) according to claim 1, wherein [symbol] denotes a double bond.

11. A compound of formula (I) according to claim 1, wherein X is bromine and Y is hydrogen, [symbol] denotes a double bond, R is hydrogen, and n is 2.

12. A compound of formula (I) according to claim 1, wherein said compound is barettin.

13. A preservation solution and/or washing solution for organs comprising the compound according to claim 1 or any pharmaceutically acceptable salt thereof.

14-16. (canceled)

17. A cosmetic treatment comprising the compound according to claim 1.

18. A food and/or feed additive comprising the compound according to claim 1.
